(19) United States
(12) Patent Application Publication
(10) Pub. No.: US 2003/0225384 A1
(43) Pub. Date: Dec. 4, 2003

(54) Absorbent article having a multi-layer absorbent structure

(75) Inventors: David L. Zenker, Neenah, WI (US); Michael B. Venturino, Appleton, WI (US); Shannon K. Melius, Appleton, WI (US); Debra Jean McDowall, Neenah, WI (US); Alan F. Schleinz, Appleton, WI (US); David A. Fell, Neenah, WI (US); Julie A. Paveletzke, Appleton, WI (US); Mark J. Beitz, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/379,210

(22) Filed: Mar. 3, 2003

Related U.S. Application Data
(60) Provisional application No. 60/383,241, filed on May 23, 2002.

(51) Int. Cl. A61F 13/15; A61F 13/20

(52) U.S. Cl. 604/367

(57) ABSTRACT

A disposable absorbent article having a longitudinal direction and a lateral direction perpendicular to the longitudinal direction. The article includes an operative, liquid impermeable backsheet, an operative, liquid permeable top sheet connected to the backsheet, and a multi-layer absorbent body positioned between the topsheet and the backsheet. The absorbent body further includes an airlaid, stabilized, first absorbent layer having a first absorbent surface having a first absorbent area, and containing a quantity of absorbent fibers, a quantity of superabsorbent material and a quantity of thermally activated binder material. The absorbent body further includes a second absorbent layer located adjacent the first absorbent layer, the second absorbent layer having a second absorbent surface having a second absorbent area, and containing a quantity of absorbent fibers and a quantity of superabsorbent material, where the first absorbent area is less than the second absorbent area.

This application claims priority from U.S. Provisional Application No. 60/383,241 filed on May 23, 2002.

### FIELD OF THE INVENTION

The present invention relates to absorbent articles, desirably disposable absorbent articles such as disposable diapers that have a distinctive absorbent body including a first stabilized absorbent layer and a second base absorbent layer.

### BACKGROUND OF THE INVENTION

It is desired that absorbent articles such as diapers, training pants, incontinence garments and incontinence pads contain and absorb bodily exudates. In the manufacture of such absorbent articles, there is continual effort to improve the performance characteristics of the article. While these articles typically have many components that may influence the performance of the article, the in-use performance of the article is often greatly influenced by its ability to absorb and retain insults with a minimal amount of leakage. Accordingly, manufacturers strive to find ways of improving the absorbent properties of these articles to improve performance.

One means of improving performance of these types of articles has been the extensive use of cellulose fluff and/or superabsorbent materials. For example, recent trends in commercial diaper designs have been to use more superabsorbent materials and less fiber in the conventional on-line formation process. As such, the resultant absorbent body can be thinner while having increased absorbent capacity.

Another method of improving the absorbent characteristics of the articles can be to utilize stabilized absorbent structures capable of providing increased tensile strength for processing as well as superior absorbent characteristics and integrity for reduced leakage in relation to traditional absorbent structures. These stabilized absorbent structures may generally be produced off-line, by a process separate from the production of the entire absorbent article.

However, many of such attempts to provide absorbent articles with improved absorbency and leakage characteristics have not been completely satisfactory. For example, despite the increase in total absorbent capacity in conventional absorbent structures resulting from the addition of larger amounts of superabsorbent material, such diapers may still suffer from leakage during use. For example, one problem resulting in leakage is the inability of the absorbent core to fully uptake liquids rapidly and completely when large amounts of liquids are discharged into the absorbent article. Another difficulty that may be encountered with such conventional absorbent structures can be the poor fluid distribution upon a sudden and localized insult. Specifically, poor distribution of such an insult through the absorbent structure may result in an elevated distortion or “hump” being produced in the absorbent structure. This localized deformation may displace the article away from the wearer, which can negatively impact the fit of the article and undesirably result in leaks. Moreover, the use of stabilized absorbent structures as the primary absorbent body in an article may be cost prohibitive due to increased raw material costs, increased processing costs, and added waste as a result of performing a pad shape cut-out.

Accordingly, despite the attempts to develop improved absorbent articles, there remains a need for absorbent articles that can provide improved absorbency characteristics, tensile strength, and a resistance to leakage and distortion upon insult. Moreover, there is a need to produce such articles in a cost-effective manner by limiting excessive raw material costs and the potential for waste. That is, there remains a need for absorbent articles that possess the performance and absorbent capabilities of a stabilized absorbent while maintaining the cost-effectiveness of conventional, unstabilized absorbent structures that are formed by the traditional on-line airformed process.

### BRIEF DESCRIPTION OF THE INVENTION

In response to the difficulties and problems discussed above, new disposable absorbent articles have been invented.

In one aspect, the present invention concerns a disposable absorbent article having a longitudinal direction and a lateral direction perpendicular to the longitudinal direction. The article includes an operative, liquid impermeable backsheet, an operative, liquid permeable top sheet connected to the backsheet, and a multi-layer absorbent body positioned between the topsheet and the backsheet. The absorbent body further includes an airlaid, stabilized first absorbent layer having a first absorbent surface having a first absorbent area, and containing a quantity of absorbent fibers, a quantity of superabsorbent material and a quantity of thermally activated binder material. The absorbent body further includes a second absorbent layer located adjacent the first absorbent layer, the second absorbent layer having a second absorbent surface having a second absorbent area, and containing a quantity of absorbent fibers and a quantity of superabsorbent material, where the first absorbent area is less than the second absorbent area.

In another aspect, the present invention concerns a disposable absorbent article having a longitudinal direction and a lateral direction perpendicular to the longitudinal direction. The article includes an operative, liquid impermeable backsheet, an operative, liquid permeable top sheet connected to the backsheet, and a multi-layer absorbent body positioned between the topsheet and the backsheet. The absorbent body further includes an airlaid, stabilized first absorbent layer having a first absorbent surface having a first absorbent area, and containing a quantity of absorbent fibers, a quantity of superabsorbent material and a quantity of thermally activated bi-component binder fiber. The absorbent body further includes a second absorbent layer located adjacent the first absorbent layer, the second absorbent layer having a second absorbent surface having a second absorbent area, and containing a quantity of absorbent fibers and a quantity of superabsorbent material, where the first absorbent area is less than 70% of the second absorbent area.

In yet another aspect, the present invention concerns a disposable absorbent article having a longitudinal direction and a lateral direction perpendicular to the longitudinal direction. The article includes an operative, liquid impermeable backsheet, an operative, liquid permeable top sheet connected to the backsheet, and a multi-layer absorbent body positioned between the topsheet and the backsheet. The absorbent body further includes an airlaid, stabilized first absorbent layer having a first absorbent surface having a first absorbent area, and containing a quantity of absorbent fibers, a quantity of superabsorbent material and a quantity of thermally activated binder material. The absorbent body further includes a second absorbent layer located adjacent the first absorbent layer, the second absorbent layer having a second absorbent surface having a second absorbent area, and containing a quantity of absorbent fibers and a quantity of superabsorbent material, where the first absorbent layer has a density of at least 0.14 grams per cubic centimeter measured at 1.4 kPa of pressure, and the first absorbent area is less than the second absorbent area.

### BRIEF DESCRIPTION OF THE DRAWINGS

The present invention will be more fully understood and further advantages will become apparent when reference is made to the following detailed description of the invention and the drawings, in which:

FIG. 1 representatively illustrates a schematic, top plan view of an inward side of an absorbent article that incorporates the absorbent body of the present invention;

FIG. 2 representatively illustrates a schematic, top plan view of an outward side of an absorbent article that incorporates the absorbent body of the present invention;

FIG. 3 representatively illustrates a longitudinal, cross-sectional, side view of the absorbent article of the present invention, taken along line 3-3 of FIG. 1;

FIG. 4 depicts an enlarged cross-sectional photograph of a first absorbent layer of the present invention illustrating the sheath and core of the binder fiber;

FIG. 5 depicts an enlarged cross-sectional photograph of a first absorbent layer of the present invention illustrating the bonding that takes place between the binder fiber and other components of the first absorbent layer;

FIG. 6 representatively illustrates a schematic, top plan view of an incontinence absorbent pad used in connection with the Cradle Test described herein.

FIGS. 7A and 7B illustrates a representative, graphical plot of the liquid distribution performance of incontinence absorbent pads of FIG. 6 when subjected to the Cradle Test described herein.

### DETAILED DESCRIPTION OF THE INVENTION

The various aspects and embodiments of the invention will be described in the context of a disposable absorbent article, such as a disposable diaper. It is, however, readily apparent that the present invention could also be employed with other articles, such as diaper pants, feminine care articles, children's training pants, incontinence pads, incontinence garments, and the like. Typically, the disposable articles are intended for limited use and are not intended to be laundered or otherwise cleaned for reuse. A disposable diaper, for example, is discarded after it has become soiled by the wearer.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

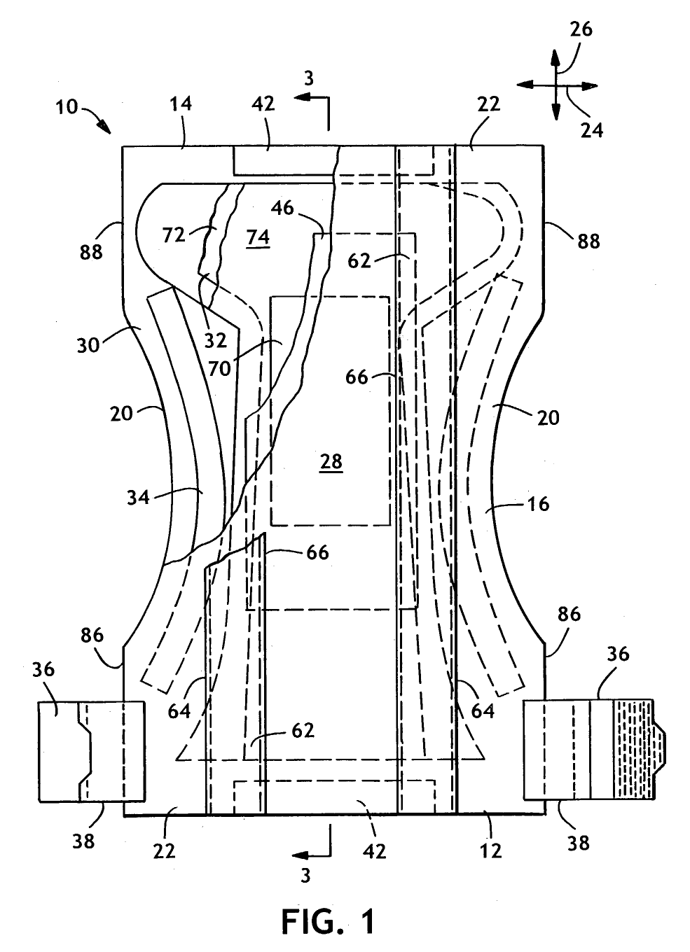

The article of the invention can, as representatively illustrated in FIGS. 1-3, be a disposable diaper 10. In desired configurations, the article can provide a first waistband portion, such as the illustrated back waistband portion 12, and a second waistband portion, such as the illustrated front waistband portion 14. The article can additionally have an intermediate or crotch portion 16 that interconnects between the first and second waistband portions 12 and 14, respectively. The article can further include a backsheet layer 30, a liquid permeable topsheet layer 28 connected and assembled in facing relation with the backsheet layer, and an absorbent structure, such as a structure which includes absorbent body 32. The absorbent structure can be sandwiched between the backsheet and topsheet layers, and is operably held therebetween. An operative fastening system, such as the illustrated system having fasteners 36, can be typically constructed and arranged to interconnect the first waistband portion 12 with the second waistband portion 14 to hold the article on a wearer. The fastening system can be operatively configured to join the first, back waistband portion 12 in an overlapping relation with the second, front waistband portion 14 in a back-to-front arrangement to thereby encircle the wearer's body and hold the diaper secure on the wearer during use. Optionally, the fastening system can employ fasteners 36 that are configured to join the front waistband portion 14 in an overlapping relation with the back waistband portion 12 in a front-to-back arrangement to secure the diaper. In such optional arrangements, the front waistband region may be identified as the first waistband portion and the rear waistband region may be identified as the second waistband portion. Alternatively, the fastening system can be provided by snaps, pins, cohesives, buckles, or any other fasteners as are well known to those skilled in the art.

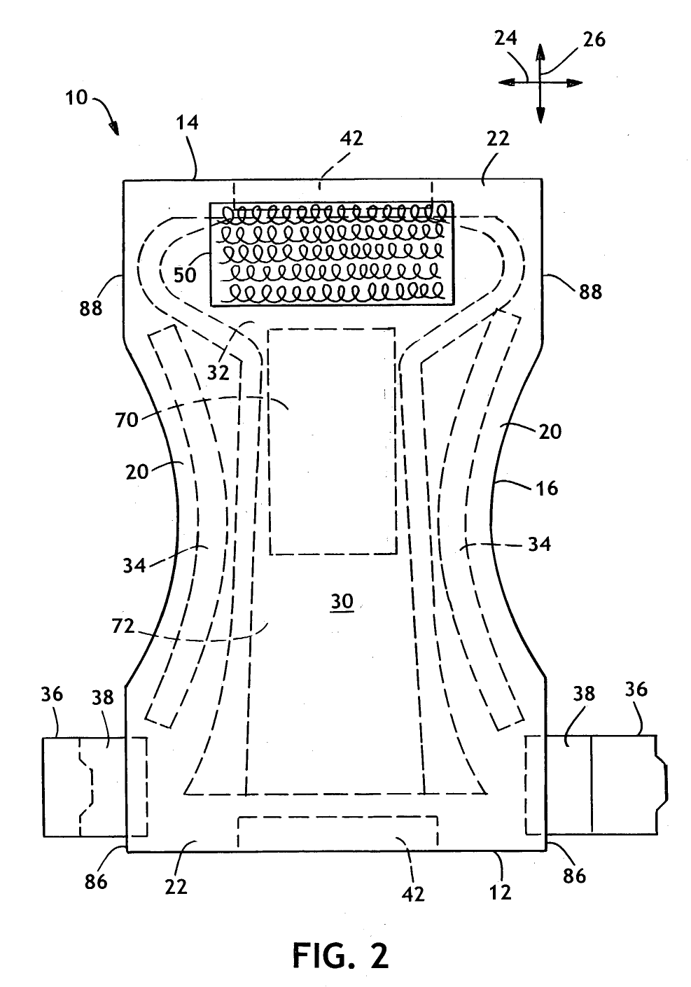

The front waistband section 14 of the representatively illustrated diaper 10 has a laterally opposed, front pair of side edge regions 88, and the rear waistband section 12 has a laterally opposed, rear pair of side edge regions 86. The intermediate section 16 interconnects the front and rear waistband section and provides a diaper crotch region which is typically positioned between the legs of the wearer. The article can also have an appointed fastener landing zone member 50 that is disposed on the outward surface of the article. In the configuration illustrated in FIG. 2, for example, the landing member 50 can be disposed on the outward surface of the backsheet layer 30. The liquid permeable topsheet layer 28 is superposed in facing relation with the backsheet layer 30, and the absorbent body 32 is operably connected and affixed between the backsheet layer 30 and topsheet layer 28.

FIGS. 1 and 2 illustrate typical plan views of the representative disposable diaper 10 in its generally flat-out, uncontracted state (i.e., with substantially all elastic induced gathering and contraction removed). In FIG. 1, portions of the structure are partially cut away to more clearly show the interior construction of the diaper article, and the bodyside surface of the diaper which contacts the wearer is facing the viewer. The outer edges of the diaper define a periphery with longitudinally extending side edge margins 20 and laterally extending end edge margins 22. The side edges define leg openings for the diaper, and optionally, are curvilinear and contoured. The end edges are illustrated as straight, but optionally, may be curvilinear.

With regard to the designated surfaces of the article, the various inward or bodyside surfaces are configured to face toward the body of the wearer when the article is placed about the wearer. The designated outward surfaces of the article are configured to face away from the wearer's body when the article is placed about the wearer.

With reference to FIGS. 1, 2 and 3, the diaper 10 can typically include a porous, liquid permeable topsheet 28 and a substantially liquid impermeable backsheet 30. In addition, the diaper of the present invention includes a distinctive absorbent body structure 32 positioned between the topsheet and backsheet. Optionally, the diaper may include a surge management portion 46 located adjacent the absorbent structure; and a system of elastomeric gathering members, such as a system including leg elastics 34 and waist elastics 42. The surge management portion is positioned in liquid communication with an appointed portion of the absorbent structure. As used herein, the term “liquid communication” is intended to represent a relationship between structures where fluid may pass from one structure to another structure. The topsheet 28, backsheet 30, absorbent structure 32, surge management portion 46 and elastic members 34 and 42 may be assembled together into a variety of well-known diaper configurations. The diaper can additionally include a system of containment flaps 62, and a system of side panel or ear region members 38, which may be elasticized or otherwise rendered elastomeric.

Examples of articles which include elasticized side panels and selectively configured fastener tabs as well as various techniques for forming the desired fastening systems are described in U.S. Pat. No. 5,399,219 of T. Roessler et al., entitled METHOD FOR MAKING A FASTENING SYSTEM FOR A DYNAMIC FITTING DIAPER which issued Mar. 21, 1995; in U.S. Pat. No. 5,540,796 entitled A PROCESS FOR ASSEMBLING ELASTICIZED EAR PORTIONS by D. Fries, which issued Jul. 30, 1996; and in U.S. Pat. No. 5,595,618 entitled AN ASSEMBLY PROCESS FOR A LAMINATED TAPE by D. Fries, which issued Jan. 21, 1997. The disclosures of the above-described documents are incorporated herein by reference in a manner that is consistent (i.e. not in conflict) herewith.

The diaper 10 generally defines the longitudinally extending length direction 26 and the laterally extending width direction 24, as representatively illustrated in FIGS. 1 and 2. The diaper may have any desired shape, such as rectangular, I-shaped, a generally hourglass shape, or a T-shape. With the T-shape, the crossbar of the “T” may comprise the front waistband portion of the diaper, or may alternatively comprise the rear waistband portion of the diaper.

The topsheet 28 and backsheet 30 may be generally coextensive, and may have length and width dimensions which are generally larger than and extend beyond the corresponding dimensions of the absorbent structure 32 to provide for the corresponding side margins 20 and end margins 22. Optionally, the topsheet and backsheet layers may not be coextensive. The topsheet 28 can be operatively associated with and superimposed on backsheet 30, thereby defining the periphery of the diaper. The waistband regions comprise those portions of the diaper, which when worn, wholly or partially cover or encircle the waist or mid-lower torso of the wearer. The intermediate, crotch region 16 lies between and interconnects the waistband regions 14 and 12, and comprises that portion of the diaper which, when worn, is positioned between the legs of the wearer and covers the lower torso of the wearer. Thus, the intermediate crotch region 16 is an area where repeated fluid insults typically occur in the diaper or other disposable absorbent articles, and may also be referred to as the “insult region” of the absorbent article.

The backsheet 30 can typically be located along an outer-side surface of the absorbent body 32 and may be composed of a vapor permeable material, but desirably comprises a material which is configured to be substantially impermeable to liquids. For example, a typical backsheet can be manufactured from a thin plastic film, or other flexible, substantially liquid-impermeable material. As used in the present disclosure, the term “flexible” refers to materials which are compliant and which will readily conform to the general shape and contours of the wearer's body. Backsheet 30 prevents the exudates contained in absorbent body 32 from wetting articles, such as bedsheets and overgarments, which contact diaper 10. In particular embodiments of the invention, backsheet 30 can include a film, such as a polyethylene film, having a thickness of from about 0.012 millimeters (0.5 mil) to about 0.051 millimeters (2.0 mil). For example, the backsheet film can have a thickness of about 0.032 millimeters (1.25 mil).

Alternative constructions of the backsheet may comprise a woven or non-woven fibrous web layer which has been totally or partially constructed or treated to impart the desired levels of liquid impermeability to selected regions that are adjacent or proximate the absorbent body. For example, the backsheet may include a gas-permeable, nonwoven fabric layer laminated to an appointed facing surface of a polymer film layer that may or may not be gas-permeable. Ordinarily, the fabric layer is attached to an outward facing surface of the polymer film layer. Other examples of fibrous, cloth-like backsheet materials can comprise a stretch-thinned or a stretch-thermal-laminate material composed of a 0.6 mil (0.015 mm) thick polypropylene blown film and a 0.7 ounce per square yard (23.8 g/m²) polypropylene spunbond material (2 denier fibers).

In particular arrangements, a substantially liquid impermeable, vapor permeable backsheet layer may be a composite material which includes a vapor permeable film layer adhesively laminated to a spunbond layer. One suitable vapor permeable film layer can be obtained from Tredegar Film Products, under the tradename EXAIRE. The film layer can include 48-60 weight percent (wt %) linear low density polyethylene and 38-50 wt % calcium carbonate particulates that may be uniformly dispersed and extruded into the film layer. The stretched film layer can have a thickness of about 0.7 mil (about 0.018 mm) and a basis weight of 16-22 grams per square meter (g/m²).

The spunbond layer can be adhesively laminated to the film layer, and can have a basis weight of about 27 g/m². The spunbond layer can be made using conventional spunbond technology, and can include filaments of polypropylene having a fiber denier of 1.5-3 dpf. The vapor-permeable film layer may be adhered to the spunbond layer using a pressure sensitive, hot melt adhesive at an add-on rate of about 1.6 g/m², and the adhesive can be deposited in the form of a pattern of adhesive swirls or a random fine fiber spray.

The liquid impermeable, vapor permeable backsheet layer may alternatively include a highly breathable stretch thermal laminate material (HBSTL). The HBSTL material can include a polypropylene spunbond material thermally attached to a stretched breathable film. For example, the HBSTL material may include a 0.6 osy (20.4 g/m²) polypropylene spunbond material thermally attached to an 18.7 g/m² stretched breathable film. The breathable film may include two skin layers with each skin layer composed of 1-3 wt % EVA/catalloy. The breathable film may also include 55-60 wt % calcium carbonate particulates, linear low density polyethylene, and up to 4.8% low density polyethylene. The stretched breathable film can include a thickness of 0.45-0.50 mils (0.011-0.013 mm) and a basis weight of 18.7 g/m². The spunbond layer can be thermally bonded to the breathable film, and can have a basis weight of about 20.4 g/m². The spunbond layer can have a fiber denier of 1.5-3 dpf, and the stretched breathable film can be thermally attached to the spunbond material using a “C-star” pattern that provides an overall bond area of 15-20%.

The various types of such materials have been employed to form the backsheet or outercover of HUGGIES disposable diapers, which are commercially available from Kimberly-Clark Corporation. The backsheet 30 typically provides the outer cover of the article. Optionally, however, the article may include a separate outer cover component member that can be additional to the backsheet. The backsheet may also be embossed or otherwise provided with a pattern or matte finish to exhibit a more aesthetically pleasing appearance.

As mentioned, the backsheet 30 may include a micro-porous, “breathable” material which permits gases, such as water vapor, to escape from the absorbent body 32 while substantially preventing liquid exudates from passing through the backsheet. For example, the breathable backsheet may be composed of a microporous polymer film or a nonwoven fabric that has been coated or otherwise modified to impart a desired level of liquid impermeability. Another example of a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn.

In still yet another alternative, the backsheet 30 may be provided by extensible or even elastic materials. For example, backsheet 30 may be configured to elongate in the lateral direction, the longitudinal direction, or in certain configurations both the lateral and longitudinal directions. Moreover, in situations where the backsheet 30 is provided by elastic materials, the backsheet may be capable of retracting within a certain degree of its original dimension after the elongating force is removed.

In the various configurations of the invention, where a component such as the backsheet 30 or the containment flaps 62 are configured to be permeable to gas while having a resistance and limited permeability to aqueous liquid, the liquid resistant material can have a construction which is capable of supporting a selected hydrohead of water substantially without leakage therethrough. For. example, desired materials can support a hydrohead of at least about 45 cm of water substantially without leakage therethrough. A suitable technique for determining the resistance of a material to liquid penetration is Federal Test Method Standard FTMS 191 Method 5514, 1978, or an equivalent thereof.

The size of the backsheet 30 is typically determined by the size of absorbent body 32 and the particular diaper design selected. Backsheet 30, for example, may have a generally T-shape, a generally I-shape or a modified hourglass shape, and may extend beyond the terminal edges of absorbent body 32 by a selected distance, such as a distance within the range of about 1.3 centimeters to 2.5 centimeters (about 0.5 to 1 inch), to provide at least a portion of the side and/or end margins.

The topsheet 28 presents a body-facing surface that is compliant, soft-feeling, and non-irritating to the wearer's skin. Further, the topsheet 28 can be less hydrophilic than absorbent body 32, and is sufficiently porous to be liquid permeable, permitting liquid to readily penetrate through its thickness to reach the absorbent body. A suitable topsheet layer 28 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The topsheet layer 28 is typically employed to help isolate the wearer's skin from liquids held in absorbent body 32.

Various woven and nonwoven fabrics can be used for topsheet 28. For example, the topsheet may be composed of a meltblown or spunbonded web of the desired fibers, and may also be a bonded-carded-web. The various fabrics can be composed of natural fibers, synthetic fibers or combinations thereof.

For the purposes of the present description, the term “nonwoven web” means a web of fibrous material that is formed without the aid of a textile weaving or knitting process. The term “fabrics” is used to refer to all of the woven, knitted and nonwoven fibrous webs.

The topsheet fabrics may be composed of a substantially hydrophobic material, and the hydrophobic material may optionally be treated with a surfactant or otherwise processed to impart a desired level of wettability and hydrophilicity. In a particular embodiment of the invention, topsheet 28 can be a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.35% surfactant. Suitable surfactants are well known in the art and include Ahcovel Base N-62 manufactured by ICI Americas, a company having offices in Bridgewater, N.J., and Glucopan 220UP manufactured by Henkel Corporation, a company having offices in Dusseldorf, Germany. The surfactant can be applied by any conventional means, such as spraying, printing, brush coating, foaming, dip and squeeze, or the like.

The topsheet 28 and backsheet 30 are connected or otherwise associated together in an operable manner. As used herein, the term “associated” encompasses configurations in which the topsheet 28 is directly joined to the backsheet 30 by affixing the topsheet 28 directly to the backsheet 30, and configurations wherein the topsheet 28 is indirectly joined to the backsheet 30 by affixing the topsheet 28 to intermediate members which in turn are affixed to the backsheet 30. Topsheet 28 and backsheet 30 can, for example, be joined to each other in at least a portion of the diaper periphery by suitable attachment mechanisms (not shown) such as adhesive bonds, ultrasonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the topsheet 28 to the backsheet 30. It should be readily appreciated that the above-described attachment mechanisms may also be employed to suitably interconnect, assemble and/or affix together the various other component parts of the articles that are described herein.

The diaper 10 can also include a surge management layer 46 which helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent structure. In the illustrated embodiment, for example, the surge layer 46 can be located on an inwardly facing body side surface of the topsheet layer 28. Alternatively, the surge layer 46 may be located adjacent to an outer side surface of the topsheet 28. Accordingly, the surge layer would then be interposed between the topsheet 28 and the absorbent body 32. Examples of suitable surge management layers 46 are described in U.S. Pat. No. 5,486,166 entitled FIBROUS NONWOVEN WEB SURGE LAYER FOR PERSONAL CARE ABSORBENT ARTICLES AND THE LIKE by C. Ellis and D. Bishop, which issued Jan. 23, 1996; and U.S. Pat. No. 5,490,846 entitled IMPROVED SURGE MANAGEMENT FIBROUS NONWOVEN WEB FOR PERSONAL CARE ABSORBENT ARTICLES AND THE LIKE by C. Ellis and R. Everett, which issued Feb. 13, 1996; the entire disclosures of which are hereby incorporated by reference in a manner that is consistent herewith.

The leg elastic members 34 are located in the lateral side margins 20 of diaper 10, and are arranged to draw and hold diaper 10 against the legs of the wearer. The elastic members are secured to diaper 10 in an elastically contractible condition so that in a normal under strain configuration, the elastic members effectively contract against diaper 10. The elastic members can be secured in an elastically contractible condition in at least two ways; for example, the elastic members may be stretched and secured while diaper 10 is in an uncontracted condition. Alternatively, diaper 10 may be contracted, for example, by pleating, and the elastic members secured and connected to diaper 10 while the elastic members are in their relaxed or unstretched condition. Still other mechanisms, such as heat-shrink elastic material, may be used to gather the garment.

In the embodiment illustrated in FIGS. 1 and 2, the leg elastic members 34 extend essentially along the complete length of the intermediate crotch region 16 of diaper 10. Alternatively, elastic members 34 may extend the entire length of diaper 10, or any other length suitable for providing the arrangement of elastically contractible lines desired for the particular diaper design.

The elastic members 34 may have any of a multitude of configurations. For example, the width of the individual elastic members 34 may be varied from about 0.25 millimeters (0.01 inch) to about 25 millimeters (1.0 inch) or more. The elastic members may comprise a single strand of elastic material, or may comprise several parallel or non-parallel strands of elastic material, or may be applied in a rectilinear or curvilinear arrangement. Where the strands are non-parallel, two or more of the strands may intersect or otherwise interconnect within the elastic member. The elastic members may be affixed to the diaper in any of several ways that are known in the art. For example, the elastic members may be ultrasonically bonded, heat and pressure sealed using a variety of bonding patterns, or adhesively bonded to diaper 10 with sprayed or swirled patterns of hotmelt adhesive.

In particular embodiments of the invention, the leg elastic members 34 may include a carrier sheet to which are attached a grouped set of elastics composed of a plurality of individual elastic strands. The elastic strands may intersect or be interconnected, or be entirely separated from each other. The carrier sheet may, for example, comprise a 0.002 cm thick polymer film, such as a film of unembossed polypropylene material. The elastic strands can, for example, be composed of LYCRA elastomer available from DuPont, a business having offices in Wilmington, Del. Each elastic strand is typically within the range of about 470-1500 decitex (dtx), and may be about 940-1050 dtx. In particular embodiments of the invention, for example, three or four strands can be employed for each elasticized legband.

In addition, the leg elastics 34 may be generally straight or optionally curved. For example, the curved elastics can be inwardly bowed toward the longitudinal centerline of the diaper. In particular arrangements, the curvature of the elastics may not be configured or positioned symmetrically relative to the lateral centerline of the diaper. The curved elastics may have an inwardly bowed and outwardly bowed, reflex-type of curvature, and the length-wise center of the elastics may optionally be offset by a selected distance toward either the front or rear waistband of the diaper to provide desired fit and appearance. In particular embodiments of the invention, the innermost point (apex) of the set of curved elastics can be offset towards the front or rear waistband of the diaper, and the outwardly bowed reflexed-portion can be positioned toward the diaper front waistband.

As representatively illustrated, the diaper 10 can include a waist elastic 42 positioned in the longitudinal margins of either or both of the front waistband 14 and the rear waistband 12. The waist elastics may be composed of any suitable elastomeric material, such as an elastomer film, an elastic foam, multiple elastic strands, an elastomeric fabric or the like. For example, suitable elastic waist constructions are described in U.S. Pat. No. 4,916,005 by Lippert et al., the entire disclosure of which is hereby incorporated by reference in a manner that is consistent herewith.

With reference to the representative configurations illustrated in FIGS. 1 and 2, the article can include a system of “ear” regions or ear members 38. In particular arrangements, each ear region or member 38 extends laterally at the opposed, lateral ends of at least one waistband portion of backsheet 30, such as the representatively illustrated rear waistband portion 12, to provide terminal side sections of the article. In addition, each ear region can substantially span from a laterally extending, terminal waistband edge to approximately the location of its associated and corresponding leg opening section of the diaper. The diaper 10, for example, has a laterally opposed pair of leg openings provided by the curved margins of the ear regions in combination with the correspondingly adjacent, medial sections of the illustrated pair of longitudinally extending, side edge regions 20 (e.g. FIG. 1).

In the various configurations of the invention, the ear regions may be integrally formed with a selected diaper component. For example, ear regions 38 can be integrally formed from the layer of material which provides backsheet layer 30, or may be integrally formed from the material employed to provide topsheet 28. In alternative configurations, the ear regions 38 may be provided by one or more separately provided members that are connected and assembled to the backsheet 30, to the topsheet 28, in between the backsheet and topsheet, or in various fixedly attached combinations of such assemblies.

In particular configurations of the invention, each of the ear regions 38 may be formed from a separately provided piece of material which is then suitably assembled and attached to the selected front and/or rear waistband portion of the diaper article. For example, each ear region 38 may be attached to the rear waistband portion of the backsheet 30 along an ear region attachment zone, and can be operably attached to either or both of the backsheet and topsheet components of the article. The inboard, attachment zone region of each ear region can be overlapped and laminated with its corresponding, lateral end edge region of the waistband section of the article. The ear regions extend laterally to form a pair of opposed waist-flap sections of the diaper, and are attached with suitable connecting means, such as adhesive bonding, thermal bonding, ultrasonic bonding, clips, staples, sewing or the like. Desirably, the ear regions extend laterally beyond the terminal side edges of the backsheet layer and topsheet layer at the corresponding, attached waistband section of the article.

The ear regions 38 may be composed of a substantially non-elastomeric material, such as polymer films, woven fabrics, nonwoven fabrics or the like, as well as combinations thereof. In particular aspects of the invention, ear regions 38 may be composed of a substantially elastomeric material, such as a stretch-bonded-laminate (SBL) material, an elastomeric neck-bonded-laminate (NBL) material, an elastomeric film, an elastomeric foam material, or the like, which is elastomerically stretchable at least along the lateral direction 24. For example, suitable meltblown elastomeric fibrous webs for forming ear regions 38 are described in U.S. Pat. No. 4,663,220 by T. Wisneski et al. which issued May 5, 1987, the entire disclosure of which is hereby incorporated by reference in a manner that is consistent herewith. Examples of composite fabrics comprising at least one layer of nonwoven textile fabric secured to a fibrous elastic layer are described in European Patent Application EP 0 217 032 A2 published on Apr. 8, 1987 which has the listed inventors of J. Taylor et al., the entire disclosure of which is hereby incorporated by reference in a manner that is consistent herewith. Examples of NBL materials are described in U.S. Pat. No. 5,226,992 by Morman which issued Jul. 13, 1993, the entire disclosure of which is hereby incorporated by reference in a manner that is consistent herewith.

As previously mentioned, various suitable constructions can be employed to attach the ear regions 38 to the selected waistband portions of the article. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753 by P. Van Gompel et al. which issued Jul. 3, 1990, the entire disclosure of which is hereby incorporated by reference in a manner that is consistent herewith.

Each of the ear regions 38 extends laterally at one of the opposed lateral ends of at least one waistband section of the diaper 10. In the illustrated embodiment, for example, a first pair of ear regions extends laterally at the opposed lateral ends of the back waistband section of the backsheet 30. Additionally, a second pair of ear regions may be included to extend laterally at the opposed lateral ends of the front waistband section of the backsheet. The illustrated ear regions have a tapered, curved or otherwise contoured shape in which the longitudinal length of the relatively inboard base region is larger or smaller than the longitudinal length of its relatively outboard end region. Alternatively, the ear regions may have a substantially rectangular shape, and optionally may have a substantially trapezoidal shape.

Diaper 10 can also include a pair of elasticized containment flaps 62 that extend generally length-wise along the longitudinal direction 26 of the diaper. The containment flaps are typically positioned laterally inboard from the leg elastics 34, and substantially symmetrically placed on each side of the lengthwise, longitudinal centerline of the diaper. In the illustrated arrangements, each containment flap 62 has a substantially fixed edge portion 64 and a substantially moveable edge portion 66, and is operably elasticized to help each containment flap to closely contact and conform to the contours of the wearer's body. Examples of suitable containment flap constructions are described in U.S. Pat. No. 4,704,116 by K. Enloe which issued Nov. 3, 1987, the entire disclosure of which is hereby incorporated by reference in a manner that is consistent herewith. The containment flaps may be composed of a wettable or a non-wettable material, as desired. In addition, the containment flap material may be substantially liquid-impermeable, may be permeable to only gas or may be permeable to both gas and liquid. Other suitable containment flap configurations are described in U.S. Pat. No. 5,562,650 by R. Everett et al., which issued Feb. 13, 1996, the disclosure of which is hereby incorporated by reference in a manner that is consistent herewith.

In optional, alternative configurations of the invention, diaper 10 may include internal, elasticized, containment waist flaps, such as those described in U.S. Pat. No. 4,753,646 issued Jun. 28, 1988, to K. Enloe; and in U.S. Pat. No. 5,904,675 by D. Laux et al., which issued May 18, 1999; the entire disclosures of which are hereby incorporated by reference in a manner that is consistent herewith. Similar to the construction of the containment flaps, the containment waist flaps may be composed of a wettable or non-wettable material, as desired. The waist flap material may be substantially liquid-impermeable, permeable to only gas, or permeable to both gas and liquid.

To provide a desired refastenable fastening system, diaper 10 can include one or more, appointed landing member regions or patches, such as provided by the representatively illustrated, primary landing member 50. The landing member can provide an operable target area for generating a releasable and re-attachable securement with at least one of the fastener tabs 36. In desired embodiments of the invention, the landing member patch can be positioned on the front waistband portion 14 of the diaper and located on the outward surface of the backsheet layer 30. Alternatively, the landing member patch can be positioned on an appointed inward surface of the diaper, such as the bodyside surface of the topsheet layer 28.

Particular arrangements of the invention can include one or more landing members 50 that can be directly or indirectly attached to the second waistband portion 14. Desirably, the landing members are affixed directly to the outward surface of the appropriate waistband portion, but may optionally be joined to the inward, bodyside surface of the article waistband portion.

In the various configurations of the invention, the landing member 50 can be composed of a substantially non-elastomeric material, such as polymer films or tapes, woven fabrics, nonwoven fabrics or the like, as well as combinations thereof. In particular configurations of the invention, the landing member may be composed of a substantially elastomeric material, such as a stretch-bonded-laminate (SBL) material, an elastomeric neck-bonded-laminate (NBL) material, an elastomeric film, an elastomeric foam material, or the like, which is elastomerically stretchable at least along the lateral direction 24.

The various configurations of the invention can include at least one separately provided fastener tab 36 located at either or both of the lateral end regions 86 of the back waistband 12. Alternatively, the at least one separately provided fastener tab 36 can be located at either or both of the lateral end regions 88 of the front waistband 14. The representatively illustrated embodiment, for example, has a laterally opposed pair of the fastener tabs 36 with a one of the fastener tabs located at each of the distal side edges of the rear waistband 12. More particularly, each of the fasteners 36 is assembled and attached to project and extend from a corresponding, immediately adjacent ear region located at one of the opposed, lateral end regions 86 of the front waistband section 12.

The fastener tab 36 can have any operative shape. For example, the shape of the fastener tab may be rectangular, trapezoidal, sinusoidal, rectilinear, curvilinear or the like, as well as combinations thereof. The laterally outboard, terminal edge of the fastener tab may be rectilinear or curvilinear, and as representatively illustrated, the terminal edge may be contoured to provide a protruding finger tab region.

The fastener tab 36 can be composed of a substantially non-elastomeric material, such as polymer films or tapes, woven fabrics, nonwoven fabrics or the like, as well as combinations thereof. Optionally, the fastener tab may be composed of a substantially elastomeric material, such as a stretch-bonded-laminate (SBL) material, a neck-bonded-laminate (NBL) material, an elastomeric film, an elastomeric foam material, or the like, which is elastomerically stretchable at least along the lateral direction 24.

In the various aspects and configurations of the invention, the fastening mechanism between the selected first fastener component and the selected, second fastener component may be adhesive, cohesive, mechanical or combinations thereof. In the context of the present invention, a mechanical fastening system is a system that includes cooperating, first and second components that mechanically inter-engage to provide a desired securement.

Desirably, the first and second fastener components include complementary elements of a cooperatively interengaging mechanical fastening system. The mechanical fastener components can be provided by mechanical-type fasteners such as hooks, buckles, snaps, buttons and the like, which include cooperating and complementary, mechanically interlocking components.

For example, the mechanical fastening system may be a hook-and-loop type of fastening system. Such fastening systems typically include attachment members having the form of a “hook” or hook-like, male component, and include a cooperating “loop” or loop-like, female component that engages and releasably interconnects with the hook component. Desirably, the interconnection is selectively releasable and re-attachable. Conventional systems are, for example, available under the VELCRO trademark. The hook element may be provided by a single-prong hook configuration, a multiple-prong hook configuration or by a generally continuous, expanded-head configuration, such as provided by a mushroom-head type of hook element. The loop element may be provided by a woven fabric, a nonwoven fabric, a knitted fabric, a perforated or apertured layer, and the like, as well as combinations thereof. The many arrangements and variations of such fastener systems have been collectively referred to as hook-and-loop fasteners.

A configuration which employs a selectively releasable, interengaging mechanical fastening system can, for example, locate the first fastener component on at least the appointed mating or securing surface of the fastener tab 36, and can locate the cooperating, second fastener component on the appointed engagement surface of the appointed landing member 50. For example, with the representatively illustrated hook-and-loop fastener, the fastening component which is attached to the appointed mating or securing surface of the fastener tab 36 may include a hook type of mechanical engagement element, and the complementary fastening component, which is operably joined and attached to the appointed landing zone member 50 can include a loop type of fastening element.

It should also be readily apparent that, in the various configurations of the invention, the relative positions and/or materials of the first fastening component and its cooperating, complementary second fastening component can be transposed. Accordingly, the fastening component, which is attached to the appointed mating surface of the fastener tabs 36, may include the loop type of mechanical fastening element; and the complementary, second fastening component, which is operatively joined and attached to the appointed landing zone member, can include the hook type of attachment members.

The absorbent article of the different aspects of the present invention also includes a distinctive absorbent body 32. The absorbent body 32 provides an absorbent structure that is configured for holding and storing absorbed liquids and other waste materials. The absorbent body can be positioned and sandwiched between the topsheet 28 and backsheet 30 to form the diaper 10. The absorbent body 32 has a construction that can be generally compressible, conformable, non-irritating to the wearer's skin, and capable of absorbing and retaining body exudates. It should be understood that, for purposes of this invention, the absorbent body structure may include a plurality of individual separate pieces of material that are operably assembled together.

For example, the absorbent body 32 may include a first absorbent layer 70 and a second absorbent layer 72. As representatively illustrated in FIGS. 1-3 the first absorbent layer can be positioned toward the interior, bodyside surface of the diaper, on the bodyside surface of the second absorbent layer 72. The first absorbent layer 70 augments the absorbent body 32 by providing improved dry and wet strength characteristics as compared to conventional airformed absorbent bodies. Moreover, the first absorbent layer 70 can be configured to have increased dry tensile strength characteristics in comparison to many conventional absorbent structures, thereby presenting improved processing capabilities. In particular, the improved wet tensile strength allows the first absorbent layer 70 to better maintain the pad shape for better fit and containment. For example, the first absorbent layer may have a wet tensile strength of at least 100 grams as determined by the tensile test set forth herein. Further, the use of the first absorbent layer 70 in the absorbent body 32 can desirably reduce the opportunity for leakage in the diaper 10.

The first absorbent layer 70 of the present invention may be constructed of any number of absorbent materials as are well known in the art. For example, the first absorbent layer may be provided by a layer of coform, meltblown fibers, bonded carded webs, tissue laminates, foams, a surge/airlaid composite and the like or combinations thereof. In particular, the first absorbent layer 70 may be provided as an airlaid pledget that can be a combination of hydrophilic fibers, high absorbency material, and binder material. As used herein, the term “airlaid” refers to the process of producing an absorbent material where unlike components are conveyed in an air-stream and homogenously mixed or provided in a stratified configuration. For example this may include, but is not limited to, a mixture of pulp fibers, synthetic fibers, superabsorbent materials and binder material. There are a number of commercial processes available to produce airlaid absorbent structures. For example airlaid processes are available from Danweb Corp. having offices in Risskov, Denmark, and from M&J Forming Technologies having offices in Horsens, Denmark.

The first absorbent layer 70 of the present invention advantageously can be presented as an airlaid stabilized absorbent. As used herein, the term “stabilized absorbent” refers to an absorbent structure that has a quantity of binder material added to a mixture of other absorbent materials, such as a quantity of absorbent fibers and a quantity of superabsorbent material, to provide an absorbent matrix. It should be noted that the absorbent mixture (for example, the quantity of absorbent fibers and quantity of superabsorbent material) and the quantity of binder material may be provided in a homogeneous mixture; or the quantity of binder material may be added to the absorbent mixture in a stratified configuration. The binder material is then activated to bond the resultant absorbent matrix together.

Stabilized airlaid absorbent structures generally offer superior performance characteristics over traditional airformed absorbent structures, as will be discussed in greater detail below. As used herein, the term “airformed” refers to absorbent structures that are produced in an unstabilized configuration. That is, an airformed absorbent matrix does not contain any activated binder material, and as such, could include other absorbent materials such as pulp fiber and high absorbency material. Accordingly, airformed absorbent structures will generally have hydrogen bonding and fiber entanglement as the primary means of maintaining the integrity of the absorbent structure.

The superior performance of absorbent structures produced by an airlaid process is the result of the ability of an airlaid process to provide uniformity of the mixture of raw materials and the ability to add synthetic fibers and/or binder material to the mixture to stabilize the resultant absorbent. As a stabilizer, binders reduce the amount of wet collapse in the structure and maintain a lower density in the saturated state. That is, the binder assists the absorbent matrix in maintaining its integrity even under load or while saturated.

Various types of wettable, hydrophilic fibrous material can be used to provide the quantity of absorbent fiber material for the first absorbent layer 70. Examples of suitable fibers include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material that has a suitable hydrophilic moiety and preferably is not readily removable from the fiber, or by sheathing the nonwettable, hydrophobic fiber with a hydrophilic polymer during or after the formation of the fiber. For the purposes of the present invention, it is contemplated that selected blends of the various types of fibers mentioned above may also be employed.

In a particular aspect where the wettable, hydrophilic fibrous material is a cellulosic fiber, the cellulosic fiber may be produced by a number of processes as are well known in the art. For example cellulosic fibers may be made by wood pulping processes that include, but are not limited to Kraft, sulphite, chemi-thermomechanical pulping (CTMP), thermomechanical pulping (TMP), or groundwood pulping. In addition, cellulosic fibers may also be bleached using suitable bleaching techniques. Sources of cellulosic fibers as described above may include, but are not limited to softwoods, hardwoods, flax, straw, and other organic materials, and combinations thereof.

The quantity of high-absorbency material used in the first absorbent layer 70 may comprise a quantity of absorbent gelling materials, such as a quantity of superabsorbent material. Absorbent gelling materials can be natural, synthetic and modified natural polymers and materials. In addition, the absorbent gelling materials can be inorganic materials, such as silica gels, or organic compounds such as cross-linked polymers. The term “cross-linked” refers to any means for effectively rendering normally water-soluble materials substantially water insoluble but swellable. Such means can include, for example cross-linking by covalent bonds. Other means include molecular entanglement, crystalline domains, ionic complexes and associations, hydrophilic associations, and hydrophobic associations.

Examples of synthetic absorbent gelling material polymers include the alkali metal and ammonium salts of poly(acrylic acid) and poly (methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further polymers suitable for use in the absorbent body include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic absorbent polymers can also be useful in the present invention. Other suitable absorbent gelling materials are disclosed by Assarsson et al. in U.S. Pat. No. 3,901,236 issued Aug. 26, 1975. Processes for preparing synthetic absorbent gelling polymers are disclosed in U.S. Pat. No. 4,076,663 issued Feb. 28, 1978 to Masuda et al. and U.S. Pat. No. 4,286,082 issued Aug. 25, 1981 to Tsubakimoto et al.

The high-absorbency material used in the first absorbent layer 70 can generally be in the form of discrete particles. The particles can be of any desired shape, for example, spiral or semi-spiral, cubic, rod-like, polyhedral, etc. Shapes having a large greatest dimension/smallest dimension ratio, like needles, flakes, and fibers, are also contemplated for use herein. Conglomerates of particles of absorbent gelling material may also be used in the first absorbent layer 70. Desired for use are particles having an average size of from about 20 micrometers to about 1 millimeter. “Particle size” as used herein means the weighted average of the smallest dimension of the individual particles.

The first absorbent layer 70 may also include a quantity of binder material such that it can be provided as a stabilized absorbent structure. Stabilization is accomplished by bonding the individual fibrous components of the absorbent structure together to create a cohesive structure. The binder material can be of a polymeric or non-polymeric binder material that is capable of forming ionic bonds, covalent bonds, or physical entanglement with the fiber and/or the high absorbency material. Moreover, the binder material may be a liquid or non-liquid binder material.

Examples of suitable polymeric binders can include polypropylene glycol (PPG); polyethylene glycol (PEG); polyacrylic acid (PAA); poly(caprolactone) diol; polyamide; cationic acrylamide copolymers; polyamine; polyamide-polyamine-epichlorohydrin (KYMENE); cationic amine-epichlorohydrin wet-strength agents; polyethylene imine agents; polyamide-epichlorohydrin agents with cellulose ethers or cationic starches for improving paper wet-strength; polyacrylamides-glyoxal (e.g. PAREZ); urea-formaldehyde agents (UF); cationic modified ureaformalin agents; melamine-formaldehyde agents (MF); cationic modified melamine-formalin agents; polyethyleneimine (PEI); dialdehyde starch (DAS); proteinaceous adhesives treated with formaldehyde; cellulose xanthate (viscose); synthetic latexes; vegetable gums such as guar and bean gum; neutral (or alkaline-curing) thermosetting wet-strength agents; water-soluble polymers containing carboxyl groups or carboxylate ions as their alkali metal or ammonium salts; substantially non-thermosetting tertiary-amino polyamide-epichlorohydrin agents.

Some commercial liquid binders are KYMENE 557LX, a polyamidoamine modified with epichlorohydrin (available from Hercules); CREPEPLUS 75, 97, a polyamidoamine modified with low epichlorohydrin content (available from Betz Paper Chemicals); CREPETROL 190, a polyamidoamine modified with low epichlorohydrin content (available from Hercules); PEI, polyethylenimine, molecular weight 50,000-60,000, 50% (wt) an aqueous liquid (available from Aldrich Chemical Co.); PEI-E a polyethylenimine modified with epichlorohydrin, base polymer mol. wt. 20,000, 17% (wt) an aqueous liquid (available from Aldrich Chemical Co.); POLYMIN PR971L, a high charge density, high molecular weight polyethylenimine (available from BASF); POLYMIN SNA, a modified high molecular weight polyethylenimine (available from BASF); and AGEFLOC WT-20VHV, a polydimethyldiallylammonium chloride (available from CPS Chemical).

Examples of non-polymeric binders can include glycerin; ascorbic acid; urea; glycine; pentaerythritol; a monosaccharide or a disaccharide; citric acid; glyoxal; tartaric acid; dipropylene glycol; and urea derivatives such as DMDHEU (dimethyldihydroxyethylurea). Suitable saccharides can include glucose, sucrose, lactose, ribose, fructose, mannose, arabinose, and erythrose.

Stabilization of the first stabilized layer 70 may also be achieved by use of emulsion binders. Physical strength can also be imparted by the use of a class of materials described herein as “latex binders.” Examples of such latex binders include, but are not limited to, emulsion polymers such as thermoplastic vinyl acetate, C1-C8 alkyl ester of acrylic, methacrylic acid based adhesive, and combinations thereof. In particular, the emulsion polymerized thermoplastic adhesive can have a Tg from −25° C., to 20° C., a solids content of from 45% to 60% by weight, typically from 52% to 57%, and a Brookfield viscosity (#4 spindle, 60 rpm at 20*C.) of from 5 to 1000 centipoises (cps). Preferred adhesives are vinyl acetate/ethylene based adhesives incorporating less than about 10% and preferably less than 5% by weight, of a polymerized third monomer. Representative examples of third monomers which may be incorporated into the polymer include adhesion. promoting monomers such as unsaturated carboxylic acid including acrylic and methacrylic acid, crotonic acid, and epoxide containing monomers such as glycidyl acrylate, glycidylmethacrylate and the like. The Airflex 401, 405 and 410 are some examples. These binders can be obtained from Air Products and Chemicals Inc. located in Allentown Pa. In addition, cross linkable binders (thermoset) may be used to impart further wet strength thereto. The thermoset vinyl acetate/ethylene binders, such as vinyl acetate/ethylene having from 1-3% N-methylolacrylamide such as Airflex 124, 108 or 192, available from Air Products and Chemicals Inc. located in Allentown, Pa., or Elite 22 and Elite 33, available from National Starch & Chemicals, located in Bridgeport, N.J., are examples of suitable adhesive binders.

To obtain a stabilization structure, emulsion polymerized thermoplastic polymeric adhesive is applied to an un-stabilized fluff/superabsorbent structure in an amount ranging from 1 to 20 grams dry adhesive per square meter of substrate. In particular aspects, 5 to 15 grams of dry adhesive per square meter of substrate where the dry adhesive is applied by a spray method may provide suitable bonds.

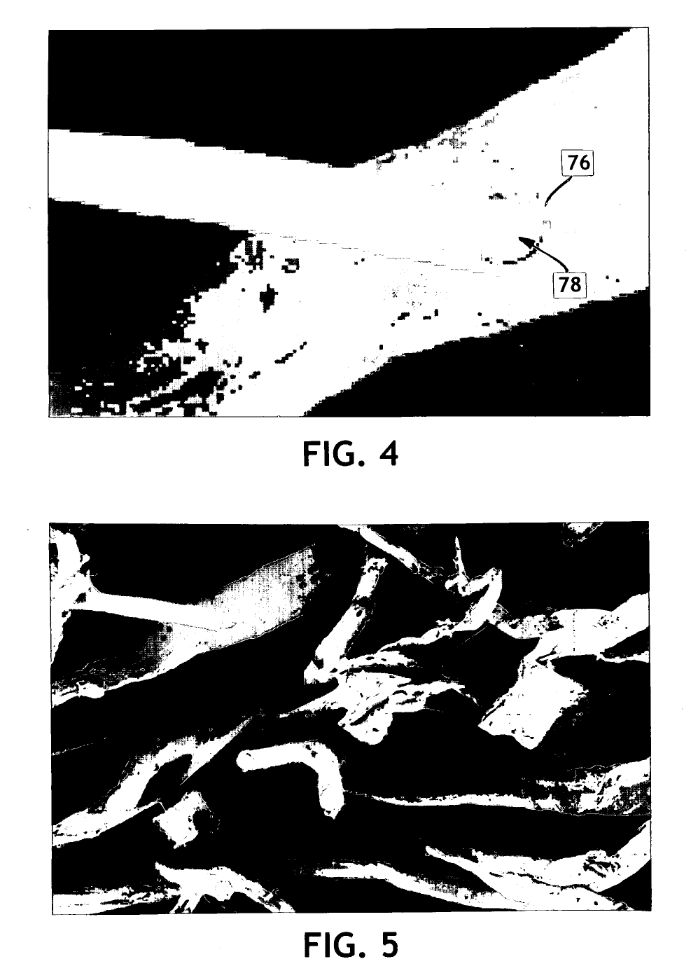

Non-liquid binder material may also be used as a stabilizing agent. For example, binder powders may be used to stabilize absorbent structures. Binder powders for use in absorbent structures are available under the trade name VINNEX available from Wacker Polymer Systems L.P., having offices in Adrian, Mich. Alternatively, thermally activated binder material, such as thermally activated binder fiber material, may be used to stabilize absorbent structures. Binder fibers are typically used in airlaid absorbent structures for higher basis weight absorbent structures, that is, greater than 120 gsm. Binder fibers generally have two components and are therefore termed bi-component fibers. Specifically, as representatively illustrated in FIG. 4, the two components include a sheath 76 and a core 78. Other suitable binder fiber configurations also include side by side, islands in the sea, and thermoplastic staple fibers. Suitable binder fibers for use in absorbent structures are available from KoSa, having offices in Houston, Tex., Chisso Corporation, having offices in Tokyo, Japan, and Trevira GmbH, having offices in Bobingen, Germany.

In particular aspects where the binder fibers are configured to be thermally activated bi-component binder fibers, the sheath 76 acts as the bonding agent in the stabilized absorbent structure. It bonds between both the core 78 of the binder fiber as well as other components in the absorbent matrix. The core 78 helps to provide resiliency, as it supplies added structural support to the absorbent matrix. The sheath 76 is often made from low density or linear low density polyethylene. To ensure necessary wettability and binding to the core 78 and other components in the absorbent, a wetting agent can be utilized. Exemplary agents are described in U.S. Pat. No. 4,578,414 issued Mar. 25, 1986 to Sawyer et al. Two types of material, polyester and polypropylene, may provide the core 78 of the bi-component fiber. Cores made of polyester typically provide higher resiliency but at a higher overall cost versus binder fiber cores that are made of polypropylene. In certain aspects, the thermally activated bi-component binder fiber may have a fiber length of at least 6 mm for improved performance.

In a particular configuration, the first absorbent layer 70 can include a uniform mixture of 50% superabsorbent material, 46% pulp fiber, and 4% bi-component binder material. For example, the superabsorbent material may be Stockhausen SXM 9543 superabsorbent material; the pulp fiber may be Caressa 1300 Pulp fiber, and the binder material may be 4% KoSa T-255 bi-component binder material. The Stockhausen superabsorbent material is available from Stockhausen Inc., a business having offices located in Greensboro, N.C., the Caressa 1300 pulp is available from Buckeye Technologies Inc., a business having offices located in Memphis, Tenn., and the KoSa binder material is available from KoSA, a business having offices located in Houston, Tex.

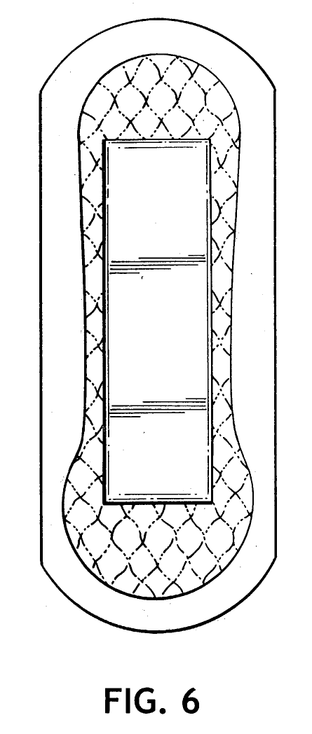

FIG. 5 representatively illustrates an enlarged cross-sectional view of the stabilized absorbent portion 70 of the present invention. In particular, the binder/binder bonding and pulp fiber/binder bonding that may occur in a stabilized absorbent structure is illustrated therein. In addition, the high absorbency material is illustrated as being trapped in the interstices between pulp and binder fibers. In some circumstances, binder fiber may also bond to high absorbency material (not shown), particularly in circumstances where the absorbent body includes higher concentrations of high absorbency material.

Bonding of the fibers as described above may be achieved by opening the binder fiber and mixing it with pulp fibers and optionally high absorbency materials. This combination is then fed through a forming head and deposited on a forming wire. The above process ensures that the absorbent materials described above have been adequately mixed. The fibrous web is then passed into an oven such that the sheath 76 of the binder fiber softens but the core 78 remains as a solid. The web can be maintained at these conditions for a given dwell time to allow the sheath 76 to flow amongst the adjacent pulp and core fibers 78. The amount of dwell time depends on the composition, basis weight and density of the web, line speed and oven temperature. The resulting web is then cooled and the sheath material 76 solidifies and partially crystallizes forming bonds with adjacent absorbent material.

Single component thermoplastic fibers can also be used to stabilize the absorbent structure (not shown). Examples of suitable single component thermoplastic fibers are well known to those skilled in the art and include polyethylene, polypropylene, and polyester fibers. Such single component fibers can be introduced as relatively short staple fibers, and then subsequently mixed with pulp fibers and optionally high absorbency materials, and finally bonded following the above process.

Because the first absorbent layer 70 of the absorbent body 32 can be stabilized by binder material as described above, the permeability of the absorbent body 32 can be increased relative to conventional absorbent structures. In addition, the dry and wet tensile strength of the first absorbent layer 70 can also be greater than that of conventional absorbents. An additional benefit of stabilization is that it reduces the amount of wet collapse experienced by the absorbent structure while in the saturated state. As such, the first absorbent layer 70 advantageously maintains “loft” so as to retain improved absorbent qualities and reduce the possibility of leakage. Thus, the amount of binder material added to an absorbent structure can influence the properties of the resultant absorbent body. In a particular aspect, the quantity of binder material in the first absorbent layer 70 can be from 3% to 15% by weight. In another aspect, the quantity of binder material in the first absorbent layer can be 4% to 10% by weight.

In addition, for improved performance, it may be advantageous to have a particular proportion of absorbent materials providing the first absorbent layer 70. Specifically, to provide the desired leakage and containment properties, while yet balancing cost considerations, the first absorbent layer 70 may be configured to have a specific percentage of superabsorbent material. For example, in one aspect, the first absorbent layer 70 may be at least 10% superabsorbent material by weight, and in particular, the first absorbent layer 70 may alternatively be at least 35% superabsorbent material by weight. In another aspect, the first absorbent layer 70 may be at least 50% to 80% superabsorbent material by weight.

As previously mentioned, the above absorbent components are combined in an airlaid process to provide the stabilized first absorbent layer. The process is desirably an off-line process for reduced manufacturing costs and improved efficiency. Alternatively, the first absorbent layer may be provided on-line, during the manufacture of the entire absorbent article. The first absorbent layer 70 is particularly adapted for off-line converting as the stabilization of the structure advantageously provides higher tensile strength than conventional absorbent structures. For example, the first absorbent layer 70 of the present invention may advantageously have a dry tensile strength of at least 3100 grams as determined by the tensile test set forth herein. In particular embodiments, the first absorbent layer may have a dry tensile strength of at least 4000 grams as determined by the tensile test set forth herein. As such, the first absorbent layer 70 thereby supplies superior integrity and web strength to facilitate off-line processing and handling. For example, the first absorbent layer 70 may be provided in the form of a continuous roll of absorbent material. The absorbent may then be unwound and introduced into the diaper manufacturing process in a continuous web. The continuous web of primary absorbent may then be cut into discreet pledgets for application into the diaper of the present invention. By producing the stabilized primary absorbent in an off-line process several advantages may be achieved. Primarily, the benefits of a completely stabilized absorbent body may be substantially realized by the present invention, while avoiding the undesirable manufacturing and material costs associated with having entirely stabilized absorbent body, since the stabilized material can be strategically placed in the absorbent article.

The first absorbent layer 70 may be provided in various shapes and sizes as are well known to those skilled in the art. For example, the first absorbent layer 70 may be rectangular, triangular, elliptical, round, or the like. Desirably, the first absorbent layer 70 can be provided in a generally rectangular shape having a particular length and width for reduced manufacturing costs and improved efficiency.

For example, the length of the first absorbent layer 70 desirably sufficiently covers the fluid loading zone in the longitudinal direction so the performance and benefits of the stabilized primary absorbent can be maximized. As used herein the term “fluid loading zone” refers to the area of the absorbent article that is most likely to receive the majority of liquid insults. In diapers, the intermediate crotch region 16 is an area where repeated fluid surges typically occur, and thus would be the fluid loading zone. However, to avoid excessive costs associated with the use of stabilized absorbents, the length in the longitudinal direction 26 of the first absorbent layer 70 is desirably kept to an effective minimum. To advantageously achieve this balance between performance and cost, the first absorbent layer length in the longitudinal direction 26 may be between 20% and 80% of the entire absorbent body length in the longitudinal direction 26. In another aspect, the first absorbent layer length may alternatively be between 30% and 70% of the entire absorbent body length in the longitudinal direction 26. In a yet another aspect, the first absorbent layer length may be between 40% and 60% of the entire absorbent body length in the longitudinal direction 26. In a particular configuration, the first absorbent layer length may be 55% of the entire absorbent body length in the longitudinal direction 26.

Likewise, the first absorbent layer width in the lateral direction 24 may similarly be advantageously sized to maximize performance and reduce costs. That is, the first absorbent layer width in the lateral direction 24 advantageously covers the fluid loading zone for improved absorption and leakage performance. Simultaneously, it is desirable to limit material and manufacturing costs associated with the first absorbent layer 70. As such, the first absorbent layer maximum width in the lateral direction 24 may be equal to or less than the absorbent body minimum width in the lateral direction 24. In such a configuration, the need to shape or remove parts of the first absorbent layer 70 is avoided. Specifically, the first absorbent layer maximum width in the lateral direction 24 can be between 25% and 100% of the absorbent body minimum width. In another aspect, the first absorbent layer maximum width can be between 50% and 100% of the absorbent body minimum width. In yet another aspect, the first absorbent layer maximum width can be between 75% and 100% of the absorbent body minimum width. In a particular configuration, the first absorbent layer maximum width can be 85% of the absorbent body minimum width. Alternatively, in another aspect, the first absorbent layer maximum width may be less than 80% of the width of the absorbent body minimum width in the lateral direction 24.

Alternatively, the first absorbent layer maximum width in the lateral direction 24 may be greater than the absorbent body minimum width in the lateral direction 24. In such a configuration, additional absorbent material is supplied in the fluid loading zone for improved leakage protection. Specifically, the first absorbent layer maximum width can be between 100 % and 150% of the absorbent body minimum width. In another aspect, the first absorbent layer maximum width can be between 100% and 125% of the absorbent body minimum width, and in a particular aspect, the first absorbent layer maximum width can be between 100% and 110% of the absorbent body 32 minimum width.

The basis weight of the first absorbent layer 70 can desirably be configured to maximize performance while managing the material costs associated with a stabilized absorbent. In particular, the basis weight of the first absorbent layer 70 may be between 100 GSM to 1500 GSM. More particularly, the basis weight of the first absorbent layer 70 may be between 300 GSM to 1000 GSM. Still more particularly, the basis weight of the first absorbent layer 70 may be between 400 GSM to 800 GSM. In a specific configuration, the basis weight of the first absorbent layer 70 can be 660 GSM.

The absorbent body 32 of the different aspects of the present invention also includes a second absorbent layer 72. As representatively illustrated in FIGS. 1-3, the second absorbent layer 72 can be positioned toward the backsheet layer 30 of the diaper 10, beneath the first absorbent layer 70. The second absorbent layer 72 provides additional fluid intake and storage capacity to the absorbent body 32, as well as a fluid distribution area to remove initial and repeated insults from the fluid loading zone. In addition, the second absorbent layer 72 can be generally larger than the first absorbent layer 70, and as such generally defines the shape and form of the absorbent body 32, and as a corollary, indirectly contributes to the shape and form of the absorbent article on a whole. Thus, the second absorbent layer 72 also contributes to the product fit during use.

The second absorbent layer 72 may be provided by any number of absorbent materials as are well known in the art. For example, the second absorbent layer 72 may be provided by a layer of airformed absorbent material, coform, meltblown fibers, bonded carded webs, a wetlaid body, tissue laminates, foams, and the like or combinations thereof. In a particular aspect, the second absorbent layer 72 may be provided by an airformed, unstabilized absorbent that may be a combination of materials that can include, but is not limited to a quantity of absorbent fibers and a quantity of superabsorbent material such as superabsorbent polymer particles.

Various types of wettable, hydrophilic fibrous material can be used to form the component parts of the second absorbent layer 72. Examples of suitable fibers include those previously described above as suitable for use with the first absorbent layer. In one aspect the second absorbent layer 72 can include Caressa 1300 pulp fiber, available from Buckeye Technologies Inc. having offices in Memphis, Tenn. In another aspect, the second absorbent layer can include NB-416 pulp fiber, available from Weyerhaeuser Corporation, having offices in Tacoma, Wash., USA.

The high-absorbency material used in the second absorbent layer may comprise gelling materials, such as superabsorbents. Absorbent gelling material can be selected from natural, synthetic, and modified natural polymers and materials. Examples of suitable natural and synthetic absorbent gelling materials include those described above in connection with the first absorbent layer. In particular, superabsorbent polymers suitable for use in the present invention are Hysorb 7050 polymer available from BASF located in Portsmouth, Va., DOW DRYTECH 2035LD polymer available from Dow Chemical Co. located in Midland, Mich. and Stockhausen Favor SXM 880 and SXM 9543 polymer available from Stockhausen Inc., located in Greensboro, N.C.

The structure of the second absorbent layer 72 can comprise a matrix of hydrophilic fibers, such as a web of cellulosic fluff, mixed with particles of high-absorbency material. In particular arrangements, the second absorbent layer 72 of the absorbent body 32 may comprise a mixture of superabsorbent hydrogel-forming particles and synthetic polymer meltblown fibers, or a mixture of superabsorbent particles with a fibrous coform material comprising a blend of natural fibers and/or synthetic polymer fibers. The superabsorbent particles may be substantially homogeneously mixed with the hydrophilic fibers, or may be nonuniformly mixed.

As previously mentioned, the hydrophilic fibers and superabsorbent material are desirably combined in an airforming process to provide the second absorbent layer. This airforming process can desirably be completed on-line, that is, during the diaper manufacturing process for ease of manufacturability. In a particular aspect, a uniform mixture of 40% Stockhausen SXM 9543 polymer and 60% Caressa 1300 pulp fiber is produced.

The second absorbent layer 72 may have any of a number of shapes. For example, the second absorbent layer 72 may be rectangular, I-shaped, hour glass-shaped, or T-shaped. In the illustrated embodiments, the second absorbent layer 72 is generally T-shaped. It is generally preferred that the absorbent body 32 is narrow in the crotch region 16 of the diaper 10. It has been found that the absorbent body 32 of the present invention is particularly useful in diaper applications when the width dimension in the crotch portion 16 is from 2.5 centimeters to 25 centimeters (1.0 to 10 inches), desirably no more than 12.7 centimeters (5.0 inches) and more desirably no more than 10.2 centimeters (4.0 inches). The narrow crotch width dimension of the second absorbent layer allows the absorbent body 32 to better fit between the legs of the wearer. It should be noted that in other applications, such as in adult incontinence products, the absorbent body 32 may effectively include other width dimensions in the crotch portion 16.

The basis weight of the second absorbent layer 72 can desirably be configured to minimize the cost of the second absorbent layer while maintaining performance characteristics and the desired shape of the absorbent body 32. In particular, the basis weight of the second absorbent layer 72 may be between 50 GSM to 1000 GSM. More particularly, the basis weight of the second absorbent layer 72 may be between 100 GSM to 700 GSM. Still more particularly, the basis weight of the second absorbent layer 72 may be between 200 GSM to 500 GSM. It should also be noted that the basis weight of the absorbent layer may not necessarily remain constant throughout the entire layer.

In order to provide the desired performance of the absorbent body 32, it may be advantageous to have a particular proportion of absorbent materials providing the second absorbent layer 72. Specifically, to provide the desired leakage and containment properties, while yet balancing cost considerations, the second absorbent layer 72 may be configured to have a specific maximum percentage of superabsorbent. For example, the second absorbent layer 72 may be less than 75% superabsorbent material by weight, and in particular, the second absorbent layer may alternatively be less than 60% superabsorbent material by weight. In a particular aspect, the second absorbent layer may be from 10% to 60% by weight superabsorbent material.

The first absorbent layer 70 and the second absorbent layer 72 together provide the absorbent body 32 of the present invention. The first absorbent layer 70 and the second absorbent layer 72 are preferably operably joined together and placed in fluid communication with each other. Optionally, a liquid permeable tissue or other woven or nonwoven web fibrous layer can be placed between in fluid communication with the first and second absorbent layers. In particular, as mentioned above, the first absorbent layer 70 can be disposed in the diaper of the present invention toward the topsheet layer 28 of the diaper 10, closest to the wearer of the article. Conversely, the second absorbent layer 72 can be configured to be located toward the backsheet layer 30 of the diaper 10.

The first absorbent layer 70 may be attached to the second absorbent layer 72 using several different methods as are well known to those skilled in the art. The attachment means desirably maintains a secure surface interface between the absorbent layers 70 and 72 and yet provide for the operable fluid transfer between the absorbent layers. For example, the first and second absorbent layers 70 and 72 may be joined using adhesive. The adhesive may be applied between the absorbents using a spray applicator, bead applicator, application roll, meltblowing the adhesive, or using a slotcoater. Further, various different adhesives may be applied such as hot-melt adhesive or latex adhesive.

Different adhesive patterns can be used to attach the first and second absorbent layers 70 and 72. For example, a swirl pattern can be utilized. These can be applied in a series of lines ranging from two lines at the edges to multiple lines for added attachment. The pattern and the number of lines can be varied and optimized depending on adhesive and absorbent material to provide aid in processing, provide contact between the absorbents and to minimize the impact on intake.

In a particular embodiment, the first and second absorbent layers 70 and 72 are joined together using National 34-5610 hot melt adhesive available from National Starch and Chemical Company, a company having offices in Bridgewater, N.J. In one embodiment, the adhesive is applied using a three swirl pattern at an add-on rate of about 0.018 grams/diaper to provide for effective attachment and fluid transfer to occur between the absorbent portions 70 and 72. Alternatively, the absorbent portions may be joined together using other bonding methods such as thermal bonding, ultrasonic bonding, and compression loading.

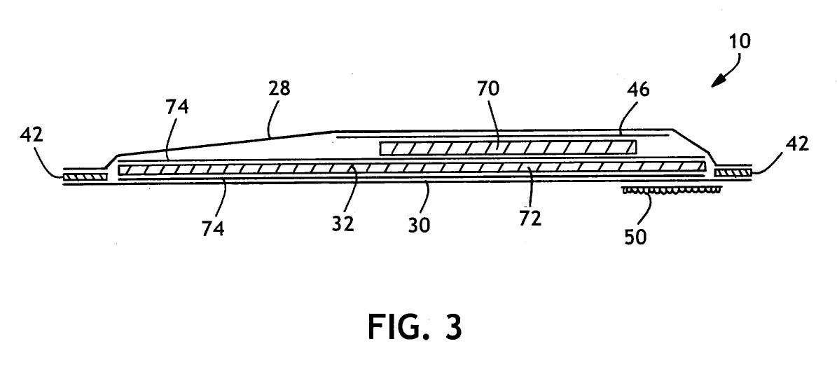

The multiple absorbent layers may be cooperatively arranged in any number of configurations as are well known in the art. For example, the absorbent portions may be layered upon one another as described above. As representatively illustrated in FIGS. 1 and 3, the first absorbent layer 70 can be located upon the second absorbent layer 72 such that it is the part of the absorbent body 32 that is closest to the wearer's body in use. Advantageously, the first absorbent layer can be located on the second absorbent layer to maximize the performance benefits associated with a stabilized absorbent body. In one aspect, as representatively illustrated in FIGS. 1-3, the primary absorbent body 70 can be located in the fluid loading zone of the diaper 10. As such, the first absorbent layer 70 is configured to be most effective for fluid intake upon insult of the diaper in use. In a particular aspect, the front edge of the first absorbent layer 70 may be located in the longitudinal direction 26 about 50 mm from front edge of the second absorbent layer 72.

In order to realize the substantial performance benefits of the composite absorbent body 32 of the present invention, the first absorbent layer 70 may provide a particular amount of the total weight of the absorbent body 32. In particular, the first absorbent layer 70 may define a first absorbent layer total weight and the second absorbent layer 72 may define a second absorbent layer total weight. The first and second absorbent layer total weights may combine to provide the absorbent body total weight. Accordingly, the contribution of the first absorbent layer total weight to the absorbent body total weight desirably balances cost considerations, the fit and comfort of the wearer due to the loft associated with the first absorbent layer 70, and its superior absorbent capabilities. For example, the first absorbent layer total weight 70 may contribute of from 10% to 90% of the absorbent body total weight. Alternatively, the first absorbent layer total weight may contribute of from 30% to 70% of the absorbent body total weight. Advantageously, the first absorbent layer total weight may contribute of from 40% to 60% of the absorbent body 32 total weight. In a desired configuration, the first absorbent layer total weight contributes 50% of the absorbent body total weight. In each of the above examples, the remainder of the absorbent body total weight is provided by the second absorbent layer total weight.

In addition, the first absorbent layer and the second absorbent layer may each have absorbent areas of various relative size configurations. For example, the first absorbent layer may define a first absorbent surface having a first absorbent area. Similarly, the second absorbent layer may define a second absorbent surface having a second absorbent area. The first and second absorbent areas may be of relative size, as is well known to those skilled in the art. For instance, the first absorbent area may be larger than the second absorbent area. Alternatively, the first absorbent area may be less than the second absorbent area. Desirably, the first absorbent area is less than the second absorbent area to provide the considerable performance benefits of the first absorbent layer while maintaining a cost sensitive absorbent structure. In a particular embodiment, the first absorbent layer may be less than 70% of the second absorbent area.

To improve the containment of the high-absorbency material, the absorbent system of the present invention can include an overwrap, such as wrap sheet 74, which can be placed immediately adjacent and around the absorbent body 32 and may be bonded to the absorbent body and to various other components of the article. The wrap sheet can desirably be liquid permeable, and can be a layer of absorbent material which covers the major bodyside and outerside surfaces of the absorbent body 32. In desired configurations, the wrap sheet can operatively enclose substantially all of the peripheral edges of the absorbent body to form a substantially complete envelope thereabout. Alternatively, the wrap sheet can provide an absorbent wrapping which covers the major bodyside and outerside surfaces of the absorbent body, and encloses substantially only the lateral side edges of the absorbent body. Accordingly, both the linear and the inwardly curved portions of the lateral side edges of the wrap sheet would be closed about the absorbent body. In such an arrangement, however, the end edges of the wrap sheet may not be completely closed around the end edges of the absorbent body at the waistband regions of the article.

For example, the wrap sheet 74, or at least the bodyside layer of the wrap sheet, may comprise a meltblown web composed of meltblown fibers, such as meltblown polypropylene fibers. Another example of the absorbent wrap 74 may comprise a low porosity cellulosic web, such as a tissue composed of an approximately 50/50 blend of hardwood/softwood fibers.

The absorbent body 32 of the different aspects of the present invention when incorporated into an absorbent article such as a diaper 10 can provide many advantages over conventional absorbent bodies as can be readily realized. For example, the stabilized first absorbent layer 70 of the absorbent body 32 supplies increased structural resistance to wet collapse, which in turn ensures that the absorbent body can provide higher composite void volume and permeability for improved absorbent intake. In addition, the first absorbent layer offers better dry and wet integrity and shape retention, giving the absorbent body of the present invention improved in-use performance and fit maintenance over conventional absorbent structures. Another advantage that the absorbent body 32 of the present invention can provide is improved fluid distribution upon a sudden and localized insult. Specifically, the absorbent structure of the present invention can reduce the incidence of an elevated distortion or “hump” resulting from a sudden insult. Further, stabilization provides improved superabsorbent particle containment versus conventional absorbent bodies. Since the stabilized absorbent portion of the absorbent body of the present invention can be configured to be the portion of the absorbent body closest to the wearer's skin, the possibility of gel migration resulting in gel on skin can also be reduced. Finally, the fact that the first absorbent layer may be cut and placed upon the second absorbent layer during manufacture provides for the ability of the absorbent body to have increased absorbent capacity more effectively located in critical areas in comparison to conventional absorbent structures.

The multilayered partially stabilized absorbent body of the present invention can also result in advantages over fully stabilized absorbent structures as well. For example, the selective placement of the stabilized first absorbent layer in critical areas of the absorbent body maximizes the utilization of the stabilized absorbent structure without incurring the considerably higher cost of a completely stabilized absorbent body. Further, the multi-layer configuration of the absorbent body of the present invention allows for greater flexibility in having a desirably shaped absorbent body through having part of the absorbent body made by conventional forming techniques. Conversely, if the absorbent body was completely of an off-line stabilized absorbent structure, providing a shaped absorbent body would be cost prohibitive as it would require substantial amounts of pad trim waste. Moreover, the use of an absorbent body that is only partially stabilized reduces the stiffness of the article in comparison to a wholly stabilized absorbent body. As such superior fit and comfort is realized.

The use of a multi-layered absorbent body including a stabilized absorbent portion can advantageously result in improved liquid intake and leakage performance with respect to conventional absorbent structures. In addition, the absorbent body of the present invention can advantageously result in improved pad integrity over conventional absorbent structures. Suitable techniques for demonstrating these improved qualities are set forth below in the “Testing” section of the present disclosure.

Testing

For Tests 1, 2 and 3 described below, samples were produced according to the following:

Example 1 included a stabilized and unstabilized absorbent, where the stabilized absorbent was positioned on top of the unstabilized absorbent. The stabilized absorbent of Example 1 was a nominally 800 GSM mix of 50% Stockhausen SXM9543 superabsorbent, available from Stockhausen Inc., 46% Caressa 1300 pulp fiber, available from Buckeye Technologies Inc., and 4% KoSa T-255 binder fiber, available from KoSA. The unstabilized absorbent of Example 1 was a nominally 400 GSM mix of 50% Stockhausen SXM 9543 superabsorbent and 50% Caressa 1300 pulp fiber. Density was targeted at 0.20 g/cc.

Comparative Example 1 included an unstabilized absorbent that was a nominally 1200 GSM mix of 50% Stockhausen SXM 9543 superabsorbent and 50% Caressa 1300 pulp fiber consisting of a first absorbent layer of nominally 800 GSM and a second absorbent layer of nominally 400 GSM. Density was targeted at 0.20 g/cc.

Comparative Example 2 included a fully stabilized absorbent that was a 1100 GSM mix of 50% Stockhausen SXM 9543 superabsorbent, 46% Caressa 1300 pulp fiber, and 4% KoSa T-255 binder fiber.

The airlaid materials in Comparative Example 1 and Comparative Example 2 were densified with a Carver press heated to 140 degrees F., a dwell time of 12 seconds, 15,000 pounds of applied force, and shims roughly equal to the target thickness of the pad based on basis weight and the 0.20 g/cc target density, measured at a pressure of 1.4 kPa.

Test 1: Absorbent Intake and Leakage Performance

Absorbent Intake Evaluation

Tests were conducted with Example 1 and Comparative Example 1 using an absorbent intake tester having separate top and bottom PLEXIGLAS, LEXAN or polycarbonate plates, each plate having a length of 17.8 cm (7.0 inches) and width of 17.8 cm (7.0 inches). The top plate of the absorbent intake tester includes a cylinder having an inner diameter of 2.5 cm (1 inch). The top plate has a circular hole formed in the center thereof.

The cylinder extends upwardly and substantially perpendicular to the surface of the top plate. The cylinder fits within the circular hole and is secured therein by an adhesive. The adhesive permanently secures the cylinder as an integral part of the top plate and prevents liquid from leaking outwardly onto the top surface of the top plate. The bottom plate has an 11.4 cm (4.5 inch) long and 11.4 cm (4.5 inch) wide piece of PLEXIGLAS, LEXAN or polycarbonate secured to the center surface of the bottom plate with adhesive. Pin elements are located near outside corners of the bottom plate. The pin elements align with apertures in the top plate to mount the plates together. A funnel is placed at the top of the cylinder to pour liquid into the test device. The combined mass of the top plate of the absorbent intake tester including the cylinder and the funnel is from 350-400 grams. The samples each had a length of 10.2 cm (4.0 inches), a width of 10.2 cm (4.0 inches).

Example 1 was placed with the stabilized absorbent in surface-to-surface relationship with the top plate of the absorbent intake tester and the unstabilized absorbent in surface-to-surface relationship with the bottom plate of the absorbent intake tester. The bottom plate and top plate of the absorbent intake tester both define substantially horizontal planes.

Both Example 1 and Comparative Example 1 were centered such that the cylinder of the top plate of the absorbent intake tester was above the center of the absorbent sample and in contact with the first absorbent layer. The test of each example began with a first insult of 60 milliliters of 0.9% aqueous saline solution being poured rapidly into the test cylinder via a funnel placed atop the cylinder. Time was recorded from first contact of solution to the absorbent sample until complete absorption had occurred (intake time in seconds). Fluid intake rate in grams per second of the absorbent sample was calculated by dividing the fluid insult amount in grams by the intake time in seconds. After a fifteen-minute wait following complete absorption of the first insult, a second 60 milliliter insult was poured into the cylinder. The time was recorded and the insult rate determined as for the first insult. A third and final insult of 60 milliliters was applied after another fifteen-minute wait following complete absorption of the second insult, and the intake time was recorded and intake rate determined.

Consumer Home Test

A Consumer Home Test is a confidential study used to evaluate in-use diaper technical performance. Panelist caregivers recruited for this study were provided with diaper test products to use on their babies. The study was conducted with approximately 225 babies that used Step 3 sized diapers intended for babies weighing 7-13 kg (16-28 lb). Approximately 50% of the babies that participated in this study were female and 50% were male. Diapers having absorbent bodies constructed approximately according to Example 1 and Comparative Example 1 were made. Each panelist was provided with sixteen diapers; eight diapers had absorbent bodies according to Example 1, and eight diapers had absorbent bodies according to Comparative Example 1. All the Example 1 diapers were to be used over a two day period. All the Comparative Example 1 diapers were to be used over a second two-day period. The sequence of use of Example 1 and Comparative Example 1 diapers was balanced between panelists. Panelists completed a diary record sheet detailing product performance after each diaper change. All of the product was collected after the completion of the test in order to maintain confidentiality.

Urine that escaped through the leg and waist openings of the diaper, thereby leaving the baby's clothing, bedding, or skin outside of the diaper wet, was considered a leak. Urine leakage frequency was calculated by dividing the number of urine leaking diapers by the total number of diapers and expressed as a percentage.

The absorbent body of Example 1 provides increased structural resistance to wet collapse, which maintains higher composite void volume and permeability over multiple insults, relative to absorbent structures according to Comparative Example 1. Table 1 illustrates that Example 1 provided superior fluid intake rates for both the second and third insults during the absorbent intake tests. In particular, Example 1 provided about a 20% improvement for intake rates over Comparative Example 1 for the second insult and about a 50% improvement for intake rates over Comparative Example 1 for the third insult. The data reflected in Table 1 reflects diapers insulted with urine only; that is, diapers insulted with a bowel movement were not included.

This improved intake functionality translated into significantly better in-use leakage performance for Example 1 compared to the diapers with absorbent bodies according to Comparative Example 1 during consumer home testing.

TABLE 1
Absorbent Intake Rate and Leakage Frequency

| | | | | Consumer |
|---|---|---|---|---|
| | | | | Home Test |
| | Absorbent Intake Rate | | | Urine |
| | Insult 1 | Insult 2 | Insult 3 | Leakage |
| | (gm/sec) | (gm/sec) | (gm/sec) | Frequency |
| Example 1 | 4.78 | 8.06 | 7.52 | 4.7% |
| Comparative | 5.30 | 6.57 | 5.03 | 7.1% |
| Example 1 | | | | |

Test 2: Pad Integrity

Edge-Wise Compression Value

The Edge-wise Compression value of Example 1, Comparative Example 1 and Comparative Example 2 were evaluated. As used herein, the Edgewise Compression Value is a measure of the dry stiffness (or flexibility) of the absorbent material. The method by which the Edge-wise Compression (EC) value can be determined is set forth in U.S. Pat. No. 6,323,388.

A 2 inch by 12 inch (5.1 cm×30.5 cm) piece of absorbent material (or product) was cut for each example, with the longer dimension aligned with the longitudinal direction of the product or raw material web. The example piece was formed into a cylinder having a height of 2 inches (5.1 cm), and with the two ends having 0-0.125 inch (0-3.18 mm) overlap, the sample is stapled together with three staples. One staple was near the middle of the width of the example, the other two nearer each edge of the width of the sample. The longest dimension of the staple was in the circumference of the formed cylinder to minimize the effect of the staples on the testing.

A Sintech tester, or similar instrument was configured with a bottom platform, a platen larger than the circumference of the sample to be tested and parallel to the bottom platform, attached to a compression load cell placed in the inverted position. The specimen was placed on the platform, under the platen. The platen is brought into contact with the specimen and compresses the sample to 50% of its width at a rate of 25 mm/min. The maximum force obtained in compressing the sample to 50% of its width (1 inch) (2.54 cm) was recorded. In addition, the energy required to compress each example to 50% of its width was recorded.

If the example buckles, it is typical for the maximum force to be reached before the sample is compressed to 50%. In an example where the length of the absorbent is less than 12 inches (30.5 cm), the Edgewise Compression Value of the material can be yet be determined. A detailed discussion of the edge-wise compression strength has been given in The Handbook Of Physical And Mechanical Testing Of Paper And Paperboard, Richard E. Mark editor, Dekker 1983, (Vol. 1 ). As discussed therein, for the Edge-wise Compression configuration described, the buckling stress is proportional to E*t²/(H²), with the proportionality constant being a function of H²/(R*t) where E is the Elastic modulus, H is the height of the cylinder, R is the radius of the cylinder, and t is the thickness of the material. When expressing the stress in terms of force per basis weight, the parameter that needs to remain constant is H²/R. Therefore, for a sample that is smaller than 12 inches (30.5 cm), the largest possible circle should be constructed and its height (width of the sample being cut out) adjusted such that H²/R equals 2.1 inches (5.3 cm).

Consumer Home Test

A Consumer Home Test is a confidential study used to evaluate in-use diaper technical performance. Panelist caregivers recruited for this study were provided with diaper test products to use on their babies. The study was conducted with approximately 225 babies that used Step 3 sized diapers intended for babies weighing 7-13 kg (16-28 lb). Approximately 50% of the babies that participated in this study were female and 50% were male. Diapers having absorbent bodies constructed approximately according to Example 1 and Comparative Example 1 were made. Each panelist was provided with sixteen diapers; eight diapers had absorbent bodies according to Example 1, and eight diapers had absorbent bodies according to Comparative Example 1. All the Example 1 diapers were to be used over a two day period. All the Comparative Example 1 diapers were to be used over a second two-day period. The sequence of use of Example 1 and Comparative Example 1 diapers was balanced between panelists. Panelists completed a diary record sheet detailing product performance after each diaper change. All of the product was collected after the completion of the test in order to maintain confidentiality.

Absorbent material deformation (including clumping and separation) occurs as a result of the physical stresses on the diaper during use. This deformation can negatively impact diaper performance, fit, and comfort. Following each use, absorbent deformation of each product was recorded. The frequency of absorbent deformation was calculated and expressed as a percentage of the total study.

Table 2 illustrates that absorbents according to Example 1 provided substantially higher dry tensile strength for improved in-use integrity with reference to Comparative Example 1. Example 1 was also more resistant to lateral compressive forces, such as caused by the action of the leg thighs of the user on the crotch region of the absorbent article, with respect to Comparative Example 1. Moreover, Example 1 provided this improved resistance to lateral compression relative to Comparative Example 1 without the increased stiffness measured in Comparative Example 2.

Finally, the improved tensile strength and resistance to lateral compression provided by Example 1 significantly reduced the in-use bunching of the absorbent bodies in diapers according to Example 1 compared to the bunching that occurred with absorbent bodies in diapers according to Comparative Example 1.

TABLE 2
Tensile, Edgewise Compression, and Absorbent Bunching

| | | | | Absorbent |
|---|---|---|---|---|
| | | Diaper Edgewise | | Bunching, |
| | Absorbent | Compression to 50% | | Separation, |
| | Tensile | Peak | | Coming |
| | Strength | Load | Energy | Apart |
| | (kg-force) | (gm) | (gm-cm) | Frequency |
| Example 1 | 4.2 | 3050 | 4570 | 31% |
| Comparative | 0.51 | 3000 | 4130 | 41% |
| Example 1 | | | | |
| Comparative | — | 5250 | 6840 | — |
| Example 2 | | | | |

For the remainder of the testing described below, samples were produced according to the following table, in addition to the previous examples:

TABLE 3

| | Basis | Superabsorbent | | |
|---|---|---|---|---|
| | Weight | material | Density | |
| Code | Gsm | Percentage | g/cc | Comments |
| A. Example Layer A | 200 | 30 | 0.14 | An Airformed absorbent |
| | | | | layer containing pulp fiber |
| | | | | and superabsorbent |
| B. Example Layer B | 400 | 30 | 0.14 | An Airformed absorbent |
| | | | | layer containing pulp fiber |
| | | | | and superabsorbent |
| C. Example Layer C | 600 | 30 | 0.14 | An Airformed absorbent |
| | | | | layer containing pulp fiber |
| | | | | and superabsorbent |
| D. Example Layer D | 200 | 30 | 0.14 | An airlaid absorbent layer |
| | | | | including 10% KoSa T255 6 |
| | | | | mm long binder fiber in |
| | | | | addition to pulp fiber and |
| | | | | superabsorbent material |
| E. Example Layer E | 400 | 30 | 0.14 | Two layers of code D |
| | | | | placed upon each other |
| F. Example Layer F | 600 | 30 | 0.14 | Three layers of code D |
| | | | | placed upon each other |
| G. Example Layer G | 200 | 30 | 0.20 | An airlaid absorbent layer |
| | | | | including 9% Trevira T255 |
| | | | | 3 mm long binder fiber in |
| | | | | addition to pulp fiber and |
| | | | | superabsorbent material |
| H. Example Layer H | 400 | 30 | 0.20 | Two layers of Code G |
| | | | | placed upon each other |
| I. Example Layer I | 600 | 30 | 0.20 | Three layers of Code G |
| | | | | placed upon each other |
| J. Example Layer J, | 600 | 25 | 0.16 | An Airformed absorbent |
| was combined with | | | | layer containing pulp fiber |
| Examples A-I to | | | | and superabsorbent |
| produce composite | | | | |
| examples | | | | |
| K. Comparative Layer K | 910 | 43.5 | 0.12 | An Airformed absorbent |
| | | | | layer containing pulp fiber |
| | | | | and superabsorbent |
| L. Comparative Layer L | 425 | 11.8 | 0.16 | An Airformed absorbent |
| combined with Example | | | | layer containing pulp fiber |
| K to produce a | | | | and superabsorbent |
| comparative composite | | | | |
| examples | | | | |

Note: Exampes A-J and Comparative example K and L used NB-416 pulp fiber. NB-416 pulp is available from Weyerhaeuser Co, Tacoma, WA. Superabsorbent was FAVOR SXM 880 available from Stockhausen, Inc.

Tensile Test

The tensile strength of absorbent samples made according to Example 1, Comparative Example 1, and Examples A, D, and G were evaluated using a Model 4201 Instron with Microcon II from the Instron Corporation, Canton Mass. The tension cell used is a 5 kilogram electronically calibrating, self-identifying load cell. The machine is calibrated and verified by hanging a series of weights within the load range of the tension cell in the center of the upper jaw, perpendicular to the jaw and hanging unobstructed. For each weight, the measured load value is compared to the actual value. Calibration, verification, and testing are performed in a room with standard-condition atmosphere such as about a temperature of about 23° C. and a relative humidity of about 50%.

For wet tensile testing the sample was soaked in 0.9% saline for 10 minutes prior to testing. A dry or wetted rectangular piece 5.1 cm (2 inches) by 15.2 cm (6 inches) of each example was placed in the pneumatic action grips Oaws). The grips had 2.5 cm (1 inch) by 7.6 cm (3 inch) rubber coated grip faces. The gauge length was 7.6 cm (3 inches) and the crosshead speed was 300 mm/minute. The crosshead speed was the rate at which the upper jaw moves upward pulling the sample until failure. The Tensile Strength value is the maximum load at failure, recorded in grams of force needed to compromise or tear each example. The results are provided and summarized in Table 4 below:

TABLE 4
Tensile Test

| | | Average Dry | | Average Wet |
|---|---|---|---|---|
| | | Tensile | | Tensile |
| | Dry Tensile Strength | Strength in | Wet Tensile Strength | Strength in |
| Example | in grams (5 Samples) | grams | in grams (5 Samples) | grams |
| G | 3074, 3148, 3095, | 3125 | 718, 809, 734, 702, | 727 |
| | 3164, 3143 | | 670 | |
| D | 4526, 4718, 4329, | 4442 | 1479, 2080, 1873, | 1980 |
| | 4234, 4404 | | 2181, 2288 | |
| A | 90, 112, 138,106, | 112 | 0-immediate failure | 0 |
| | 112 | | prevented | |
| | | | measurement | |

Test 4: Cradle Test

Material or product was placed in an acrylic cradle to simulate body curvature of a user such as an infant. Such a cradle is described in U.S. Pat. No. 5,843,063 and is illustrated in FIG. 2 of that patent. The material was insulted with 0.9 weight percent sodium chloride solution at a rate of 8-10 cc/sec with a nozzle normal to the center of the material positioned about 3 mm from the surface of the material. The test procedure was conducted as follows:

1) Measure the total absorbent length of the product.

2) Divide the total length by two (to obtain the longitudinal center of the product).

3) Cradle Position: Measure 1 cm longitudinally backward from the center of the pad and draw a line across the pad.

4) Insult Site: Measure 5 cm longitudinally forward from the line described in step 3, and place a small dot in the lateral center of the pad.

5) Record the weight the pad dry.

6) Take the bulk in the center of the dry pad with a load of 1.4 kPa.

7) Take an X-Ray of the dry pad (Always place pad in the same direction for each scanned image).

8) Attach the product in the cradle so that the product lays flat against the surface of the cradle.

9) Insult the product with 20 gms of room temperature saline at 8-10 cc/sec in the cradle.

10) If runoff occurs, record the amount and place the fluid back into the pad immediately.

11) After 20 minutes weigh the wet product.

12) Take an X-Ray of the wet pad and return to the cradle.

13) A total of at least three insults should be added to the pad, 20-25 minutes apart, with weights and x-rays after each insult.

14) Take the wet bulk of the sample as described in step 6.

15) Determine fluid distribution in the pad by x-ray image analysis.

X-ray imaging is known in the art. as discussed, for example, in an article entitled “Liquid Distribution: comparison of X-ray Imaging Data” by David F. Ring, Oscar Lijap and Joseph Pascente in Nonwovens World magazine, summer 1995, at pages 65-70. Generally, this procedure compares x-ray images of a wet and dry sample in order to calculate the liquid content. Such x-ray systems are available from Tronix Inc. of 31 Business Park Drive, Branford, Conn. 06045 as model no. 10561 HF 100 w/enclosure. This system uses software from Optumus Inc., of Ft. Collins, Colo. as Bio-scan Optimate version 4.11 (part number OPM4101105461).

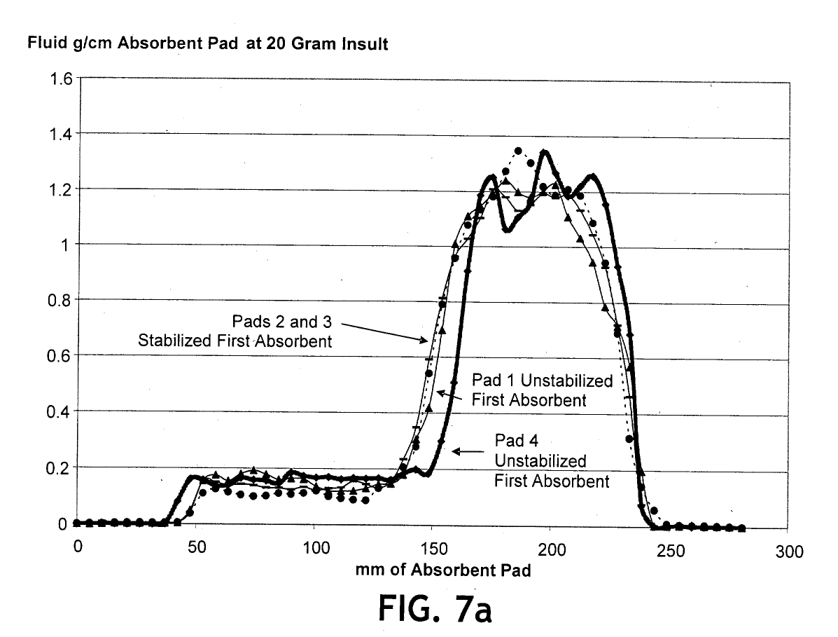

Incontinence pads representatively illustrated in FIG. 6 were made having absorbent bodies including the Example layers described in Table 3. Specifically, four incontinence pads were made and tested for liquid distribution having absorbent bodies as described below:

Pad 1: Example layer C placed upon Example layer J

Pad 2: Example layer I placed upon Example Layer J

Pad 3: Example layer F placed upon Example Layer J

Pad 4: Example layer K placed upon Example Layer L

As representatively illustrated in FIG. 6, the first absorbent had a rectangular area of 82 cm\hat{ }2 (48 mm by 172 mm) and the second absorbent had a dogbone shape with a length of 254 mm and an area of 179 cm\hat{ }2. The first and second absorbents were sandwiched between a 17 gsm spunbonded polypropylene bodyside liner material (over the first absorbent) and a water impermeable polypropylene backsheet. There was also a 50 gsm, 82 cm\hat{ }2 polypropylene surge material (density=0.04 g/cc) between the topsheet and the first absorbent. All pads had similar liquid absorption capacity of about 220 grams (measured by immersing the pad in 0.9% saline for 20 minutes, then squeezing out excess liquid under a 3.45 kPa load for 5 minutes, then subtracting the dry pad weight from the wet pad weight).

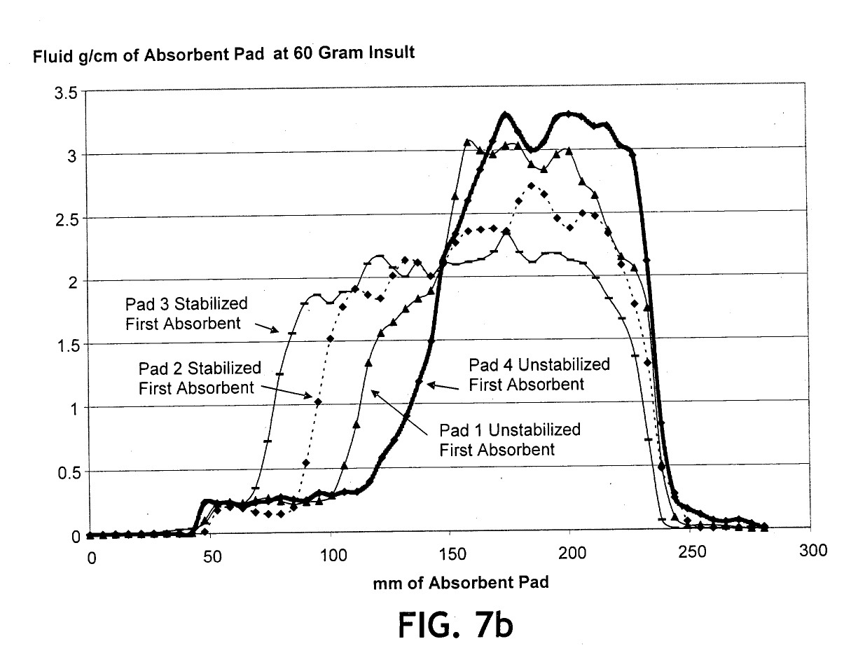

The dry pads were insulted with 20 grams of 0.9% saline solution according to the cradle procedure described above and X-Rayed. The resulting liquid distribution is illustrated in FIG. 7A. Note that only Pad 4 exhibits a difference in liquid distribution from the other three. The cradle and X-Ray procedure was then repeated for a second and a third 20 gram insult. Following the third 20 gram insult, pads 2 and 3 did not form a “hump” in the insult region of the pad despite having the same liquid absorption capacity as pads 1 and 4 (which did form a “hump”). This is illustrated in FIG. 7B, where the more uniform distribution of liquid along the full length of pads 2 and 3 is apparent. That is, the peak grams/cm loading for pads 1 and 4 with non-stabilized first absorbent layers at about the 150 to 225 mm region (the insult region for the pads) is over 3.0 g/cm compared to about 2.5 grams/cm for pads 3 and 4 with the stabilized airlaid first absorbents. Thus the configuration of pads 2 and 3 are capable or reducing saturation in the center of the pad. The stabilization of the first absorbent layer in pads 2 and 3 combined with their relatively high density (greater than 0.13 g/cc) is responsible for the improved, more uniform, distribution of liquid after insulting with 60 grams of saline. Also, the first absorbent in pads 2 and 3 contain superabsorbent (about 30%) which enables their first absorbent layer to also function as a retention, as well as a distribution, material while reducing the chance of an uncomfortable mass forming in the center of the pad as liquid is absorbed. In particular, the swelling of the center of Pads 1 and 2 were compared after 60 grams of saline insult.

Having described the invention in rather full detail, it will be readily apparent that various changes and modifications can be made without departing from the spirit of the invention. All of such changes and modifications are contemplated as being within the scope of the invention as defined by the subjoined claims.

What is claimed is:

1. A disposable absorbent article defining a longitudinal direction and a lateral direction perpendicular to said longitudinal direction, said article comprising:
an operative, liquid impermeable backsheet;
an operative, liquid permeable top sheet connected to said backsheet; and
a multi-layer absorbent body positioned between said topsheet and said backsheet, said absorbent body further comprising:
an airlaid, stabilized, first absorbent layer defining a first absorbent surface having a first absorbent area, and containing a quantity of absorbent fibers, a quantity of superabsorbent material and a quantity of thermally activated binder material; and
a second absorbent layer located adjacent said first absorbent layer, said second absorbent layer defining a second absorbent surface having a second absorbent area, and containing a quantity of absorbent fibers and a quantity of superabsorbent material, wherein said first absorbent area is less than said second absorbent area.

2. The absorbent article of claim 1 wherein said quantity of thermally activated binder material is a thermally activated binder fiber.

3. The absorbent article of claim 2 wherein said thermally activated binder fiber is a bi-component binder fiber.

4. The absorbent article of claim 1 wherein said first absorbent area is less than 70% of said second absorbent area.

5. The absorbent article of claim 1 wherein said first absorbent layer of said absorbent body is disposed toward said topsheet and said second absorbent layer of said absorbent body is disposed toward said backsheet.

6. The absorbent article of claim 1 wherein said first absorbent layer of said absorbent body is disposed toward said backsheet and said second absorbent layer of said absorbent body is disposed toward said topsheet.

7. The absorbent article of claim 1 wherein:
said first absorbent layer further defines a first absorbent layer total weight,
said second absorbent layer further defines a second absorbent layer total weight, and
said first absorbent layer total weight and said second absorbent layer total weight combine to provide an absorbent body total weight;
and wherein said first absorbent layer total weight is from 30% to 70% of said absorbent body total weight.

8. The absorbent article of claim 1 wherein:
said absorbent body defines an absorbent body length in said longitudinal direction; and
said first absorbent layer defines a first absorbent layer length in said longitudinal direction;
and wherein said first absorbent layer length is from 30% to 70% of said absorbent body length.

9. The absorbent article of claim 1 wherein:
said absorbent body defines an absorbent body minimum width in said lateral direction; and
said first absorbent layer defines a first absorbent layer maximum width in said lateral direction;
and wherein said first absorbent layer maximum width is less than 85% of said absorbent body minimum width.

10. The absorbent article of claim 1 wherein said first absorbent layer is attached to said second absorbent layer by an adhesive.

11. The absorbent article of claim 10 wherein said adhesive is applied in a swirl pattern.

12. The absorbent article of claim 1 wherein said quantity of absorbent fibers, said quantity of superabsorbent material and said quantity of thermally activated binder fiber of said first absorbent layer is provided in a homogeneous mixture.

13. The absorbent article of claim 1 wherein said basis weight of said first absorbent layer is from 100 GSM to 1500 GSM.

14. The absorbent article of claim 1 wherein said quantity of thermally activated binder fiber in said first absorbent layer is from 4% to 10% by weight.

15. The absorbent article of claim 1 wherein said quantity of superabsorbent material in said first absorbent layer is at least 10% by weight.

16. The absorbent article of claim 1 wherein said quantity of superabsorbent material in said first absorbent layer is at least 35% by weight.

17. The absorbent article of claim 1 wherein said quantity of superabsorbent material in said first absorbent layer is from 50% to 80% by weight.

18. The absorbent article of claim 1 wherein said first absorbent layer has a dry tensile strength of at least 3100 grams as determined by the tensile test set forth herein.

19. The absorbent article of claim 1 wherein said first absorbent layer has a wet tensile strength of at least 100 grams as determined by the tensile test set forth herein.

20. A disposable absorbent article defining a longitudinal direction and a lateral direction perpendicular to said longitudinal direction, said article comprising:
an operative, liquid impermeable backsheet;
an operative, liquid permeable top sheet connected to said backsheet; and
a multi-layer absorbent body positioned between said topsheet and said backsheet, said absorbent body further comprising:
an airlaid, stabilized, first absorbent layer defining a first absorbent surface having a first absorbent area, and containing a quantity of absorbent fibers, a quantity of superabsorbent material and a quantity of thermally activated bi-component binder fiber; and
a second absorbent layer located adjacent said first absorbent layer, said second absorbent layer defining a second absorbent surface having a second absorbent area, and containing a quantity of absorbent fibers and a quantity of superabsorbent material, wherein said first absorbent area is less than 70% of said second absorbent area.

21. The absorbent article of claim 20 wherein said quantity of thermally activated binder fiber in said first absorbent layer is from 4% to 10% by weight.

22. The absorbent article of claim 20 wherein:
said first absorbent layer further defines a first absorbent layer total weight,
said second absorbent layer further defines a second absorbent layer total weight,
and said first absorbent layer total weight and said second absorbent layer total weight combine to provide an absorbent body total weight;
and wherein said first absorbent layer total weight is from 30% to 70% of said absorbent body total weight.

23. The absorbent article of claim 20 wherein:
said absorbent body defines an absorbent body length in said longitudinal direction; and
said first absorbent layer defines a first absorbent layer length in said longitudinal direction;
and wherein said first absorbent layer length is from 30% to 70% of said absorbent body length.

24. The absorbent article of claim 20 wherein:
said absorbent body defines an absorbent body minimum width in said lateral direction; and
said first absorbent layer defines a first absorbent layer maximum width in said lateral direction;
and wherein said first absorbent layer maximum width is less than 85% of said absorbent body minimum width.

25. The absorbent article of claim 20 wherein said quantity of superabsorbent material in said first absorbent layer is at least 10% by weight.

26. The absorbent article of claim 20 wherein said quantity of superabsorbent material in said first absorbent layer is at least 35% by weight.

27. The absorbent article of claim 20 wherein said quantity of superabsorbent material in said first absorbent layer is from 50% to 80% by weight.

28. The absorbent article of claim 20 wherein said first absorbent layer has a dry tensile strength of at least 3100 grams as determined by the tensile test set forth herein.

29. The absorbent article of claim 20 wherein said first absorbent layer has a wet tensile strength of at least 100 grams as determined by the tensile test set forth herein.

30. A disposable absorbent article defining a longitudinal direction and a lateral direction perpendicular to said longitudinal direction, said article comprising:
an operative, liquid impermeable backsheet;
an operative, liquid permeable top sheet connected to said backsheet; and
a multi-layer absorbent body positioned between said topsheet and said backsheet, said absorbent body further comprising:
an airlaid, stabilized, first absorbent layer defining a first absorbent surface having a first absorbent area, and containing a quantity of absorbent fibers, a quantity of superabsorbent material and a quantity of thermally activated bi-component binder fibers; and
a second absorbent layer located adjacent said first absorbent layer, said second absorbent layer defining a second absorbent surface having a second absorbent area, and containing a quantity of absorbent fibers and a quantity of superabsorbent material, wherein said first absorbent layer has a density of at least 0.14 grams per cubic centimeter and said first absorbent area is less than said second absorbent area measured at a pressure of 1.4 kPa.

31. The absorbent article of claim 30 wherein said thermally activated binder fibers define a fiber length of at least 6 mm.

32. The absorbent article of claim 30 wherein said first absorbent layer has a density of at least 0.20 grams per cubic centimeter measured at a pressure of 1.4 kPa.

33. The absorbent article of claim 32 wherein said thermally activated binder fibers define a fiber length of at least 3 mm.
